(19) United States
(12) Patent Application Publication
(10) Pub. No.: US 2019/0365699 A1
(43) Pub. Date: Dec. 5, 2019

(54) Treatment of Kidney Tumors by Intratumoral Injection of Taxane Particles

(71) Applicant: CRITITECH, INC., Lawrence, KS (US)

(72) Inventors: Michael BALTEZOR, Lawrence, KS (US); Gere DIZEREGA, Lawrence, KS (US); Charles J. DECEDUE, Lawrence, KS (US); Sam CAMPBELL, Lawrence, KS (US); Matthew MCCLOREY, Lawrence, KS (US); Holly MAULHARDT, Lawrence, KS (US)

(21) Appl. No.: 16/239,531

(22) Filed: Jan. 4, 2019

Related U.S. Application Data
(60) Provisional application No. 62/678,700, filed on May 31, 2018.
(60) Provisional application No. 62/740,495, filed on Oct. 3, 2018.
(60) Provisional application No. 62/678,470, filed on May 31, 2018.
(60) Provisional application No. 62/740,489, filed on Oct. 3, 2018.
(60) Provisional application No. 62/779,327, filed on Dec. 13, 2018.

(51) Int. Cl. A61K 31/337; A61K 9/00; A61P 35/04

(52) U.S. Cl. CPC A61K 31/337; A61K 35/15; A61P 35/04; A61K 9/0019

(57) ABSTRACT

Disclosed herein are compositions and methods for treating kidney tumors by intratumoral injection administration of compositions comprising taxane particles such as docetaxel particles or paclitaxel particles.

This application claims priority to U.S. Provisional Patent Application Ser. No. 62/678,700 filed May 31, 2018; 62/740,495 filed Oct. 3, 2018; 62/678,470 filed May 31, 2018, 62/740,489 filed Oct. 3, 2018, and 62/779,327 filed Dec. 13, 2018; each incorporated by reference herein in its entirety.

### FIELD OF THE DISCLOSURE

The present disclosure generally relates to treatment of kidney tumors.

### BACKGROUND

Tumors that form in the kidneys can be malignant (cancerous) or benign (non-cancerous). Renal cell carcinoma (RCC), aka renal cell cancer or renal cell adenocarcinoma, is the most common type of kidney cancer (malignant tumor) accounting for about 90% of kidney cancers. RCC generally grows as a single tumor within a kidney, but sometimes 2 or more tumors grow in one or both kidneys. Subtypes of RCC include clear cell renal cell carcinoma which is the most common form of RCC, papillary renal cell carcinoma, chromophobe renal cell carcinoma, collecting duct RCC, multilocular cystic RCC, medullary carcinoma, mucinous tubular and spindle cell carcinoma, and neuroblastoma-associated RCC. Some types of RCCs are considered unclassified because they don't fit into any of the other RCC categories. Other types of kidney cancer include transitional cell carcinoma aka urothelial carcinoma, Wilms tumor (nephroblastoma), and renal sarcoma. Some kidney tumors are benign (non-cancerous) and include renal adenoma, oncocytoma, and angiomyolipoma.

According to the American Cancer Society, kidney cancer cells usually do not respond well to chemotherapy, so chemotherapy is not a standard treatment for kidney cancer, and whenever possible, surgery is the main treatment option. Surgery options include radical nephrectomy or partial nephrectomy. Risks and side effects of surgery include reactions to anesthesia, excess bleeding, blood clots, infections, damage to internal organs and blood vessels during surgery, pneumothorax, incisional hernia, leakage of urine into the abdomen (after partial nephrectomy), and kidney failure (if the remaining kidney fails to function well). Surgery also requires a hospital stay. If surgery is not a not an option, such as for people who are too sick to have surgery, ablation techniques can sometimes be used, such as cryoablation and radiofrequency ablation. However, according to the American Cancer Society, these ablation techniques are not considered a standard treatment. These ablation techniques can also produce side effects such as bleeding and damage to the kidneys and other nearby organs. Thus, effective treatments with less side effects and risks are needed.

### SUMMARY

The present disclosure provides solutions to the aforementioned limitations and deficiencies in the art relating to treatment of kidney tumors. Disclosed herein are compositions and methods for treating kidney tumors. The kidney tumors responded well to intratumoral injection of compositions comprising taxane particles. This was unexpected because kidney tumors are not known to respond well to chemotherapy, and chemotherapy is not a standard treatment of care for kidney tumors.

In one aspect of the disclosure, disclosed is a method of treating a kidney tumor in a subject, the method comprising administering an effective amount of a composition comprising taxane particles to a kidney tumor of the subject via intratumoral injection, wherein the taxane particles have a mean particle size (number) of from 0.1 microns to 5 microns thereby treating the kidney tumor. In some embodiments, the administering comprises two or more separate administrations. In some embodiments, the administering comprises two or more separate administrations once a week for at least two weeks. In other embodiments, the administering comprises two or more separate administrations twice a week for at least one week, wherein the two or more separate administrations are separated by at least one day. In some embodiments, the administering comprises two or more separate administrations at or at least 1, 2, 3, 4, 5, 6, 7, 8, 9, 10, 11, 12, or 14 days apart. In some embodiments, the administering comprises two or more separate administrations 2 to 12 weeks apart. In some embodiments, the composition is administered in two to five separate administrations of the composition. In some embodiments, the administering comprises 1, 2, 3, 4, 5, or 6 separate administrations of the composition. In other embodiments, the administering comprises 7 or more separate administrations of the composition.

In some embodiments, the treating of the tumor comprises elimination (eradication) of the tumor. In some embodiments, the taxane particles have a mean particle size (number) of from 0.1 microns to 1.5 microns, or from 0.4 microns to 1.2 microns. In some embodiments, the taxane particles comprise paclitaxel particles, docetaxel particles, cabazitaxel particles, or combinations thereof. In some embodiments, the taxane particles comprise at least 95% of the taxane. In some embodiments, the taxane particles are paclitaxel particles. In some embodiments the taxane particles are docetaxel particles. In some embodiments, the taxane particles and/or the docetaxel particles have a specific surface area (SSA) of at least 18 m²/g. In some embodiments, the taxane particles and/or the docetaxel particles have a bulk density (not-tapped) of 0.05 g/cm³ to 0.15 g/cm³. In some embodiments, the taxane particles are not bound to, encapsulated in, or coated with one or more of a monomer, a polymer (or biocompatible polymer), a protein, a surfactant, or albumin. In some embodiments, the taxane particles are in crystalline form. In some embodiments, the composition further comprises a liquid carrier. In some embodiments, the composition comprises a suspension of the taxane particles dispersed in the liquid carrier. In some embodiments, the kidney tumor is malignant. In further embodiments, the malignant kidney tumor is renal cell carcinoma. In other embodiments, the kidney tumor is benign.

Another aspect is that the methods of the disclosure also allow for exposure of the taxane particles to a kidney tumor after administration of the composition for a sustained amount of time sufficient to stimulate the endogenous immune system of the subject resulting in the production of tumoricidal cells and infiltration of the tumoricidal cells in and/or around the tumor site at a level sufficient to treat the tumor. In some embodiments, the stimulation of the endogenous immune systems produces a cellular (cell-mediated) immune response. In other embodiments, the stimulation of the endogenous immune system produces a humoral immune response. In some embodiments, metastases are reduced or eliminated. In some embodiments, the tumoricidal cells comprise dendritic cells, macrophages, T-cells, B-cells, lymphocytes, or natural killer (NK) cells, or combinations thereof. In some embodiments, the exposure time is at least 4 weeks. In some embodiments, the sustained amount of exposure time is at least 108, 120, 132, 144, 156, 168, 180, 192, 204, 216, 228, 240, 252, 264, 276, 288, 300, 312, 324, or 336 hours. In various further embodiments, the sustained amount of exposure time is at least 3, 4, 5, 6, 7, or 8 weeks.

Also, disclosed in the context of the present disclosure are the following embodiments 1 to 40:

Embodiment 1 is a method of treating a kidney tumor in a subject, the method comprising administering an effective amount of a composition comprising taxane particles to a kidney tumor of the subject via intratumoral injection, wherein the taxane particles have a mean particle size (number) of from 0.1 microns to 5 microns thereby treating the kidney tumor.
Embodiment 2 is the method of embodiment 1, wherein the administering comprises two or more separate administrations.
Embodiment 3 is the method of embodiments 1 or 2, wherein the administering comprises two or more separate administrations once a week for at least two weeks.
Embodiment 4 is the method of embodiments 1 or 2, wherein the administering comprises two or more separate administrations twice a week for at least one week, and wherein the two or more separate administrations are separated by at least one day.
Embodiment 5 is the method of embodiments 1 or 2, wherein the administering comprises two or more separate administrations 2 to 12 weeks apart.
Embodiment 6 is the method of any one of embodiments 1-5, wherein the administering comprises two to five separate administrations of the composition.
Embodiment 7 is the method of any one of embodiments 1-6, wherein the treating comprises elimination of the tumor.
Embodiment 8 is the method of any one of embodiments 1-7, wherein the taxane particles have a mean particle size (number) of from 0.1 microns to 1.5 microns, or from 0.4 microns to 1.2 microns.
Embodiment 9 is the method of any one of embodiments 1-8, wherein the taxane particles comprise paclitaxel particles, docetaxel particles, cabazitaxel particles, or combinations thereof. Embodiment 10 is the method of embodiment 9, wherein the taxane particles comprise at least 95% of the taxane.
Embodiment 11 is the method of any one of embodiments 1-10, wherein the taxane particles comprise paclitaxel particles.
Embodiment 12 is the method of embodiment 11, wherein the paclitaxel particles have a specific surface area (SSA) of at least 18 m²/g, 20 m²/g, 25 m²/g, 30 m²/g, 32 m²/g, 34 m²/g, or 35 m²/g.
Embodiment 13 is the method of embodiment 11, wherein the paclitaxel particles have a specific surface area (SSA) of between about 18 m²/g to about 50 m²/g.
Embodiment 14 is the method of any one of embodiments 11 to 13, wherein the paclitaxel particles have a bulk density (not-tapped) of 0.05 g/cm³ to 0.15 g/cm³.
Embodiment 15 is the method of any one of embodiments 1-10, wherein the taxane particles comprise docetaxel particles.
Embodiment 16 is the method of embodiment 15, wherein the docetaxel particles have a specific surface area (SSA) of at least 18 m²/g, 20 m²/g, 25 m²/g, 30 m²/g, 35 m²/g, 40 m²/g, or 42 m²/g.
Embodiment 17 is the method of embodiment 15, wherein the docetaxel particles have a specific surface area (SSA) of between about 18 m²/g and about 60 m²/g.
Embodiment 18 is the method of any one of embodiments 15 to 17, wherein the docetaxel particles have a bulk density (not-tapped) of 0.05 g/cm³ to 0.15 g/cm³.
Embodiment 19 is the method of any one of embodiments 1-18, wherein, the taxane particles are not bound to, encapsulated in, or coated with one or more of a monomer, a polymer (or biocompatible polymer), a protein, a surfactant, or albumin.
Embodiment 20 is the method of any one of embodiments 1-19, wherein the taxane particles are in crystalline form.
Embodiment 21 is the method of any one of embodiments 1 to 20, wherein the composition further comprises a liquid carrier, and wherein the composition comprises a suspension of the taxane particles dispersed in the liquid carrier.
Embodiment 22 is the method of embodiment 21, wherein the liquid carrier is an aqueous carrier.
Embodiment 23 is the method of embodiment 22, wherein the aqueous carrier comprises normal saline solution.
Embodiment 24 is the method of any one of embodiments 22 or 23, wherein the aqueous carrier comprises a surfactant and/or ethanol.
Embodiment 25 is the method of embodiment 24, wherein the aqueous carrier comprises a surfactant, and wherein the surfactant is a polysorbate.
Embodiment 26 is the method of embodiment 25, wherein the polysorbate is polysorbate 80, and wherein the polysorbate 80 is present in the liquid carrier at a concentration of about 0.01% w/v to about 1% w/v.
Embodiment 27 is the method of any one of embodiments 24 to 26, wherein the aqueous carrier comprises ethanol, and wherein the ethanol is present at a concentration of about 0.1% w/v to about 8% w/v.
Embodiment 28 is the method of any one of embodiments 21 to 27, wherein the composition further comprises a diluent, wherein the liquid carrier and the diluent form a mixture, and wherein the composition is a suspension of the taxane particles dispersed in the liquid carrier/diluent mixture.
Embodiment 29 is the method of embodiment 28, wherein the diluent is a normal saline solution.
Embodiment 30 is the method of any one of embodiments 21-29, wherein the taxane particles are paclitaxel particles and wherein the concentration of the paclitaxel in the composition is between about 0.1 mg/mL and about 40 mg/mL, or about 10 mg/mL and about 30 mg/mL.
Embodiment 31 is the method of any one of embodiments 21-29, wherein the taxane particles are docetaxel particles and wherein the concentration of the docetaxel particles in the composition is between about 0.1 mg/mL and about 40 mg/mL, or about 10 mg/mL and about 30 mg/mL. Embodiment 32 is the method of any one of embodiments 1-31, wherein the kidney tumor is benign.
Embodiment 33 is the method of any one of embodiments 1-31, wherein the kidney tumor is malignant.
Embodiment 34 is the method of embodiment 33, wherein the kidney tumor comprises renal cell carcinoma.
Embodiment 35 is the method of any one of embodiments 1-34, wherein the taxane particles reside at the tumor site after administration of the composition exposing the tumor to the taxane particles for a sustained amount of time sufficient to stimulate the endogenous immune system of the subject resulting in the production of tumoricidal cells and infiltration of the tumoricidal cells in and/or around the tumor site at a level sufficient to treat the tumor.
Embodiment 36 is the method of embodiment 35, wherein the stimulation of the endogenous immune system produces a cellular immune response.
Embodiment 37 is the method of embodiment 35, wherein the stimulation of the endogenous immune system produces a humoral immune response.
Embodiment 38 is the method of any one of embodiments 35-37, wherein the sustained amount of time is at least 4 weeks.
Embodiment 39 is the method of any one of embodiments 35 to 38, wherein the tumoricidal cells comprise dendritic cells, macrophages, T-cells, B-cells, lymphocytes, or natural killer (NK) cells, or combinations thereof.
Embodiment 40 is the method of any one of embodiments 1-39, wherein treating the kidney tumor comprises reducing or eliminating metastases.

It is contemplated that any embodiment discussed in this specification can be implemented with respect to any method or composition of the disclosure, and vice versa. Furthermore, compositions of the disclosure can be used to achieve methods of the disclosure.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### BRIEF DESCRIPTION OF THE FIGURES

FIG. 1 is a photomicrograph at 6.3× magnification of a histology slide from a female rat. H&E—Untreated.

FIG. 2 is a photomicrograph at 6.3× magnification of a histology slide from a female rat. H&E—Vehicle Control (IT), 3 weekly intratumoral administration cycles.

FIG. 3 is a photomicrograph at 6.3× magnification of a histology slide from a female rat. H&E—Docetaxel Solution (IV), 3 weekly IV administration cycles.

FIG. 4 is a photomicrograph at 6.3× magnification of a histology slide from a female rat. H&E—nDoce (IT), 3 weekly intratumoral administration cycles.

FIG. 5 are various photomicrographs of Control Cases. Top row: H&E stained sections. Bottom row: Immunohistochemical staining.

FIG. 6 are various photomicrographs of Intratumoral nDoce cases. Top row: One cycle nDoce (1×). Second row: One cycle nDoce (1×). Third row: Two cycles nDoce (2×). Fourth row: Two cycles nDoce (2×). Fifth row: Three cycles nDoce (3×).

FIG. 7 is a graph of mean tumor volumes vs. time of female rats in the nPac group.

FIG. 8 is a graph of mean tumor volumes vs. time of female rats in the nDoce group.

FIG. 9 is a graph of mean tumor volumes vs. time of male rats in the nDoce group.

FIG. 10 is graph of mean tumor volumes vs. time of male and rats in the nDoce group.

### DETAILED DESCRIPTION

Disclosed herein are compositions and methods for treating kidney tumors. The methods comprise the intratumoral injection of a composition comprising taxane particles directly into the kidney tumor. Surprisingly, malignant kidney tumors responded well to the intratumoral injection of compositions comprising taxane particles. This was unexpected because malignant kidney tumors are not known to respond well to chemotherapy, and chemotherapy is not a standard treatment of care for kidney tumors. Although not bound by theory, it is hypothesized that when a composition comprising taxane particles (including but not limited to paclitaxel particles or docetaxel particles) is administered intratumorally to a kidney tumor, the taxane particles will persist at the tumor site for long periods of time, e.g. greater than 4 days, or at least 14 days, or at least 4 weeks. The benefits of administering compositions comprising taxane particles into kidney tumors by intratumoral injection include the effective localized treatment of the tumors without the risks and side effects of surgical and ablation techniques, plus the treatment does not require a hospital stay.

Another benefit of the methods disclosed herein is that the exposure of the taxane particles to a kidney tumor after administration of the composition for a sustained amount of time is sufficient to stimulate the endogenous immune system resulting in (1) the production of tumoricidal cells, such as dendritic cells, macrophages, T-cells, B cells, lymphocytes, or natural killer (NK) cells, and (2) infiltration of these tumoricidal cells in and/or around the tumor site inducing tumor destruction. In some embodiments, the sustained amount of exposure time is at least 4 weeks. In some embodiments, the sustained amount of exposure time is at least 108, 120, 132, 144, 156, 168, 180, 192, 204, 216, 228, 240, 252, 264, 276, 288, 300, 312, 324, or 336 hours. In various further embodiments, the sustained amount of exposure time is at least 3, 4, 5, 6, 7, or 8 weeks. Without being limited to any specific mechanism, such effect may comprise, for example, providing sufficient time for lymphocytes to activate both their innate as well as adaptive immunological response to the tumor. Without being limited to any specific mechanism, local tumor cell killing by the administration of taxane particles intratumorally into the kidney tumor releases tumor cell antigens which are attached to dendritic cells. The activated dendritic cells may then present tumor-specific antigen to T-cells and other tumoricidal cells that circulate throughout the patient's vascular system as well as enter tissues that contain tumor allowing for destruction of cancer throughout the patient. Thus, methods disclosed herein allow for direct local therapy, as well as indirect immune system-mediated local and systemic cancer cell killing. For example, the methods disclosed herein provide the taxane molecules to act as an adjuvant to stimulate the immune response. Local concentration of taxane remains elevated for greater than 4 days, or at least 14 days, or at least 4 weeks, which provides sufficient time for the tumor to be exposed to the taxane for killing of local tumor cells as well as stimulation of the immune response appropriate for killing of cancer that may be widely disseminated through the body. This stimulation of the immune system by local administration of taxane particles occurs without producing concomitant high levels of taxane in the patient's circulating blood. Thus, local administration of particle taxane does not reduce hematopoiesis in the bone marrow involving reduction in white blood cell numbers such as lymphocytes. Bone marrow suppression is a common side effect of taxanes when given IV due to the high concentrations of circulating taxane. Thus, intratumorally administering the taxane particles is in effect a tumor vaccine given its effect in stimulating the endogenous immune system. It is also contemplated that the intratumoral injection methods disclosed herein can stimulate the endogenous immune system to produce tertiary lymphoid structures that infiltrate in and around the tumor site inducing tumor destruction. Secondary lymphoid organs develop as part of a genetically preprogrammed process during embryogenesis and primarily serve to initiate adaptive immune response providing a location for interactions between rare antigen-specific naïve lymphocytes and antigen-presenting cells draining from local tissue. Organogenesis of secondary lymphoid tissues can also be recapitulated in adulthood during de novo lymphoid neogenesis of tertiary lymphoid structures (TLSs) and form in the inflamed tissue afflicted by various pathological conditions, including cancer. Organogenesis of mucosal-associated lymphoid tissue such as bronchial-associated lymphoid tissue is one such example. The term TLS can refer to structures of varying organization, from simple clusters of lymphocytes, to sophisticated, segregated structures highly reminiscent of secondary lymphoid organs. A notable difference between lymph nodes and TLSs is the that where lymph nodes are encapsulated, TLSs represent a congregation of immune and stromal cells confined within an organ or tissue.

In some embodiments, the stimulation of the endogenous immune systems produces a cellular (cell-mediated) immune response. In other embodiments, the stimulation of the endogenous immune system produces a humoral immune response. In some embodiments, metastases are reduced or eliminated.

As used herein, the term “kidney” means one or both kidneys.

As used herein, the term “kidney tumor(s)” means a solid tumor that is an abnormal mass of tissue that is found in or on one or both kidneys, and/or in or on one or both adrenal glands. Kidney tumors may be benign (not cancer) or malignant (cancer).

As used herein, the terms “treat”, “treatment”, “treated”, or “treating” with respect to kidney tumors means accomplishing one or more of the following: (a) reducing tumor size; (b) reducing tumor growth; (c) reducing or limiting development and/or spreading of metastases, or eliminating metastases; (d) reducing or limiting development of one or more side effects of IV chemotherapy treatment; (e) eliminating a tumor. Side effects of IV chemotherapy treatment include, but are not limited to anemia, neutropenia, thrombocytopenia, neurologic toxicities, reduction in appetite, constipation, diarrhea, hair loss, fatigue, nausea/vomiting, and pain.

As used herein, the terms “intratumoral injection”, “intratumoral administration”, or “intratumoral injection administration” means that some or all of the composition, such as a suspension, is directly injected into a kidney tumor mass, and can include one or more injections at one or more injection sites in the tumor in a single administration (cycle) of a single dose. Multiple doses of the composition can be administered in two or more separate administrations. As will be understood by those of skill in the art, such direct injection may include injection of some portion of the composition on the periphery of the kidney tumor (“peritumorally”), such as if the dose amount of composition or suspension thereof is too large to all be directly injected into the solid tumor mass. In one embodiment, the composition or suspension thereof is injected in its entirety into the kidney tumor mass.

As used herein, the term “suspension” means a suspension dosage form composition where taxane particles are dispersed (suspended) within a continuous carrier or a continuous carrier/diluent mixture. The taxane particles can be completely dispersed, partially dispersed and partially dissolved, but not completely dissolved in the carrier or carrier/diluent mixture.

The terms “subject” or “patient” as used herein mean a vertebrate animal. In some embodiments, the vertebrate animal can be a mammal. In some embodiments, the mammal can be a primate, including a human.

The term “room temperature” (RT) as used herein, means 15-30° C. or 20-25° C.

The term “surfactant” or “surface active agent” as used herein, means a compound or a material or a substance that exhibits the ability to lower the surface tension of water or to reduce the interfacial tension between two immiscible substances.

As used herein, the singular forms “a”, “an” and “the” include plural referents unless the context clearly dictates otherwise. “And” as used herein is interchangeably used with “or” unless expressly stated otherwise.

The terms “about” or “approximately” as used herein mean+/−five percent (5%) of the recited unit of measure.

For this application, a number value with one or more decimal places can be rounded to the nearest whole number using standard rounding guidelines, i.e. round up if the number being rounded is 5, 6, 7, 8, or 9; and round down if the number being rounded is 0, 1, 2, 3, or 4. For example, 3.7 can be rounded to 4.

Unless the context clearly requires otherwise, throughout the description and the claims, the words “comprise”, “comprising”, and the like are to be construed in an inclusive or open-ended sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application. The compositions and methods for their use can “comprise,” “consist essentially of,” or “consist of” any of the ingredients or steps disclosed throughout the specification. With respect to the phrase “consisting essentially of,” a basic and novel property of the methods of the present disclosure is their ability to treat kidney tumors by intratumoral administrations of compositions of taxane particles into kidney tumors.

### Taxane Particles

Taxanes are poorly water-soluble compounds generally having a solubility of less than or equal to 10 mg/mL in water at room temperature. Taxanes are widely used as antineoplastic agents and chemotherapy agents. The term “taxanes” as used herein include paclitaxel (I), docetaxel (II), cabazitaxel (III), and any other taxane or taxane derivatives, non-limiting examples of which are taxol B (cephalomannine), taxol C, taxol D, taxol E, taxol F, taxol G, taxadiene, baccatin III, 10-deacetylbaccatin, taxchinin A, brevifoliol, and taxuspine D, and also include pharmaceutically acceptable salts of taxanes.

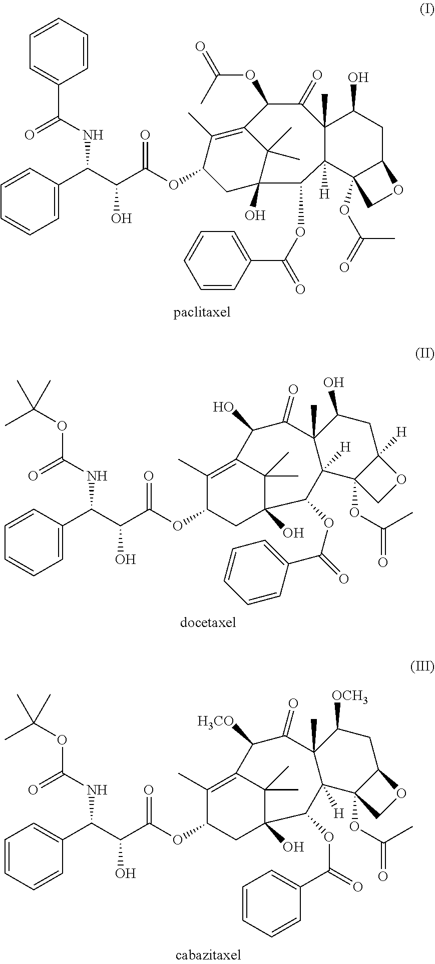

Paclitaxel and docetaxel active pharmaceutical ingredients (APIs) are commercially available from Phyton Biotech LLC, Vancouver, Canada. The docetaxel API contains not less than 90%, or not less than 95%, or not less than 97.5% docetaxel calculated on the anhydrous, solvent-free basis. The paclitaxel API contains not less than 90%, or not less than 95%, or not less than 97% paclitaxel calculated on the anhydrous, solvent-free basis. In some embodiments, the paclitaxel API and docetaxel API are USP and/or EP grade. Paclitaxel API can be prepared from a semisynthetic chemical process or from a natural source such as plant cell fermentation or extraction. Paclitaxel is also sometimes referred to by the trade name TAXOL, although this is a misnomer because TAXOL is the trade name of a solution of paclitaxel in polyoxyethylated castor oil and ethanol intended for dilution with a suitable parenteral fluid prior to intravenous infusion. Taxane APIs can be used to make taxane particles. The taxane particles are solid particles. The taxane particles can be paclitaxel particles, docetaxel particles, or cabazitaxel particles, or particles of other taxane derivatives, including particles of pharmaceutically acceptable salts of taxanes.

Taxane particles have a mean particle size (number) of from about 0.1 microns to about 5 microns (about 100 nm to about 5000 nm) in diameter. In some embodiments, the taxane particles have a mean particle size (number) of from about 0.1 microns to about 1.5 microns (about 100 nm to about 1500 nm) in diameter. In some embodiments, the taxane particles have a mean particle size (number) of from about 0.1 microns to less than micron (about 100 nm to less than 1000 nm) in diameter. In preferred embodiments, the taxane particles are solid, uncoated (“neat” or “naked”) individual particles. In some embodiments, the taxane particles are not bound to any substance. In some embodiments, no substances are absorbed or adsorbed onto the surface of the taxane particles. In some embodiments, the taxane or taxane particles are not encapsulated, contained, enclosed or embedded within any substance. In some embodiments, the taxane particles are not coated with any substance. In some embodiments, the taxane particles are not microemulsions, nanoemulsions, microspheres, or liposomes containing a taxane. In some embodiments, the taxane particles are not bound to, encapsulated in, or coated with a monomer, a polymer (or biocompatible polymer), a protein, a surfactant, or albumin. In some embodiments, a monomer, a polymer (or biocompatible polymer), a protein, a surfactant, or albumin is not absorbed or adsorbed onto the surface of the taxane particles. In some embodiments, the taxane particles exclude albumin. In some embodiments, the taxane particles are paclitaxel particles and exclude albumin. In some embodiments, the taxane particles are in crystalline form. In other embodiments, the taxane particles are in amorphous form, or a combination of both crystalline and amorphous form. In some embodiments, the taxane particles of the disclosure contain traces of impurities and byproducts typically found during preparation of the taxane. In some embodiments, the taxane particles comprise at least 90%, at least 95%, at least 96%, at least 97%, at least 98%, at least 99% or 100% of the taxane, meaning the taxane particles consist of or consist essentially of substantially pure taxane.

The taxane particles (including but not limited to paclitaxel particles, docetaxel particles, or cabazitaxel particles) can have a mean particle size (number) of from 0.1 microns to 5 microns, or from 0.1 microns to 2 microns, or from 0.1 microns to 1.5 microns, or from 0.1 microns to 1.2 microns, or from 0.1 microns to 1 micron, or from 0.1 microns to less than 1 micron, or from 0.1 microns to 0.9 microns, or from 0.1 microns to 0.8 microns, or from 0.1 to 0.7 microns, or from 0.2 microns to 5 microns, or from 0.2 microns to 2 microns, or from 0.2 microns to 1.5 microns, or from 0.2 microns to 1.2 microns, or from 0.2 microns to 1 micron, or from 0.2 microns to less than 1 micron, or from 0.2 microns to 0.9 microns, or from 0.2 microns to 0.8 microns, or from 0.2 microns to 0.7 microns, or from 0.3 microns to 5 microns, or from 0.3 microns to 2 microns, or from 0.3 microns to 1.5 microns, or from 0.3 microns to 1.2 microns, or from 0.3 microns to 1 micron, or from 0.3 microns to less than 1 micron, or from 0.3 microns to 0.9 microns, or from 0.3 microns to 0.8 microns, or from 0.3 microns to 0.7 microns, or from 0.4 microns to 5 microns, or from 0.4 microns to 2 microns, or from 0.4 microns to 1.5 microns, or from 0.4 microns to 1.2 microns, or from 0.4 microns to 1 micron, or from 0.4 microns to less than 1 micron, or from 0.4 microns to 0.9 microns, or from 0.4 microns to 0.8 microns, or from 0.4 microns to 0.7 microns, or from 0.5 microns to 5 microns, or from 0.5 microns to 2 microns, or from 0.5 microns to 1.5 microns, or from 0.5 microns to 1.2 microns, or from 0.5 microns to 1 micron, or from 0.5 microns to less than 1 micron, or from 0.5 microns to 0.9 microns, or from 0.5 microns to 0.8 microns, or from 0.5 microns to 0.7 microns, or from 0.6 microns to 5 microns, or from 0.6 microns to 2 microns, or from 0.6 microns to 1.5 microns, or from 0.6 microns to 1.2 microns, or from 0.6 microns to 1 micron, or from 0.6 microns to less than 1 micron, or from 0.6 microns to 0.9 microns, or from 0.6 microns to 0.8 microns, or from 0.6 microns to 0.7 microns.

The particle size of the taxane particles can be determined by a particle size analyzer instrument and the measurement is expressed as the mean diameter based on a number distribution (number). A suitable particle size analyzer instrument is one which employs the analytical technique of light obscuration, also referred to as photozone or single particle optical sensing (SPOS). A suitable light obscuration particle size analyzer instrument is the ACCUSIZER, such as the ACCUSIZER 780 SIS, available from Particle Sizing Systems, Port Richey, Fla. Another suitable particle size analyzer instrument is one which employs laser diffraction, such as the Shimadzu SALD-7101.

Taxane particles can be manufactured using various particle size-reduction methods and equipment known in the art. Such methods include, but are not limited to conventional particle size-reduction methods such as wet or dry milling, micronizing, disintegrating, and pulverizing. Other methods include “precipitation with compressed anti-solvents” (PCA) such as with supercritical carbon dioxide. In various embodiments, the taxane particles are made by PCA methods as disclosed in U.S. Pat. Nos. 5,874,029, 5,833,891, 6,113,795, 7,744,923, 8,778,181, 9,233,348, 9,814,685; US publications US 2015/0375153, US 2016/0374953; and international patent application publications WO 2016/197091, WO 2016/197100, and WO 2016/197101; all of which are herein incorporated by reference.

In PCA particle size reduction methods using supercritical carbon dioxide, supercritical carbon dioxide (anti-solvent) and solvent, e.g. acetone or ethanol, are employed to generate uncoated taxane particles as small as 0.1 to 5 microns within a well-characterized particle-size distribution. The carbon dioxide and solvent are removed during processing (up to 0.5% residual solvent may remain), leaving taxane particles as a powder. Stability studies show that the paclitaxel particle powder is stable in a vial dose form when stored at room temperature for up to 59 months and under accelerated conditions (40° C./75% relative humidity) for up to six months.

Taxane particles produced by various supercritical carbon dioxide particle size reduction methods can have unique physical characteristics as compared to taxane particles produced by conventional particle size reduction methods using physical impacting or grinding, e.g., wet or dry milling, micronizing, disintegrating, comminuting, microfluidizing, or pulverizing. As disclosed in U.S. Pat. No. 9,233,348, herein incorporated by reference, such unique characteristics include a bulk density (not tapped) between 0.05 g/cm³ and 0.15 g/cm³ and a specific surface area (SSA) of at least 18 m²/g of taxane (e.g., paclitaxel and docetaxel) particles, which are produced by the supercritical carbon dioxide particle size reduction methods described in U.S. Pat. No. 9,814,685 and as described below. This bulk density range is generally lower than the bulk density of taxane particles produced by conventional means, and the SSA is generally higher than the SSA of taxane particles produced by conventional means. These unique characteristics result in significant increases in dissolution rates in water/methanol media as compared to taxanes produced by conventional means. As used herein, the “specific surface area” (SSA) is the total surface area of the taxane particle per unit of taxane mass as measured by the Brunauer-Emmett-Teller (“BET”) isotherm by the following method: a known mass between 200 and 300 mg of the analyte is added to a 30 mL sample tube. The loaded tube is then mounted to a Porous Materials Inc. SORPTOMETER®, model BET-202A. The automated test is then carried out using the BETWIN® software package and the surface area of each sample is subsequently calculated. As will be understood by those of skill in the art, the “taxane particles” can include both agglomerated taxane particles and non-agglomerated taxane particles; since the SSA is determined on a per gram basis it takes into account both the larger agglomerated and smaller non-agglomerated taxane particles in the composition. The agglomerated taxane particles are defined herein as individual taxane particles that are formed by the agglomeration of smaller particles which fuse together forming the larger individual taxane particles, all of which occurs during the processing of the taxane particles. The BET specific surface area test procedure is a compendial method included in both the United States Pharmaceopeia and the European Pharmaceopeia. The bulk density measurement can be conducted by pouring the taxane particles into a graduated cylinder without tapping at room temperature, measuring the mass and volume, and calculating the bulk density.

As disclosed in U.S. Pat. No. 9,814,685, studies showed a SSA of 15.0 m²/g and a bulk density of 0.31 g/cm³ for paclitaxel particles produced by milling paclitaxel in a Deco-PBM-V-0.41 ball mill using a 5 mm ball size, at 600 RPM for 60 minutes at room temperature. Also disclosed in U.S. Pat. No. 9,814,685, one lot of paclitaxel particles had a SSA of 37.7 m²/g and a bulk density of 0.085 g/cm³ when produced by a supercritical carbon dioxide method using the following method: a solution of 65 mg/mL of paclitaxel was prepared in acetone. A BETE MicroWhirl® fog nozzle (BETE Fog Nozzle, Inc.) and a sonic probe (Qsonica, model number Q700) were positioned in the crystallization chamber approximately 8 mm apart. A stainless steel mesh filter with approximately 100 nm holes was attached to the crystallization chamber to collect the precipitated paclitaxel particles. The supercritical carbon dioxide was placed in the crystallization chamber of the manufacturing equipment and brought to approximately 1200 psi at about 38° C. and a flow rate of 24 kg/hour. The sonic probe was adjusted to 60% of total output power at a frequency of 20 kHz. The acetone solution containing the paclitaxel was pumped through the nozzle at a flow rate of 4.5 mL/minute for approximately 36 hours. Additional lots of paclitaxel particles produced by the supercritical carbon dioxide method described above had SSA values of: 22.27 m²/g, 23.90 m²/g, 26.19 m²/g, 30.02 m²/g, 31.16 m²/g, 31.70 m²/g, 32.59 m²/g, 33.82 m²/g, 35.90 m²/g, 38.22 m²/g, and 38.52 m²/g.

As disclosed in U.S. Pat. No. 9,814,685, studies showed a SSA of 15.2 m²/g and a bulk density of 0.44 g/cm³ for docetaxel particles produced by milling docetaxel in a Deco-PBM-V-0.41 ball mill using a 5 mm ball size, at 600 RPM for 60 minutes at room temperature. Also disclosed in U.S. Pat. No. 9,814,685, docetaxel particles had a SSA of 44.2 m²/g and a bulk density of 0.079 g/cm³ when produced by a supercritical carbon dioxide method using the following method: A solution of 79.32 mg/mL of docetaxel was prepared in ethanol. The nozzle and a sonic probe were positioned in the pressurizable chamber approximately 9 mm apart. A stainless steel mesh filter with approximately 100 nm holes was attached to the pressurizable chamber to collect the precipitated docetaxel particles. The supercritical carbon dioxide was placed in the pressurizable chamber of the manufacturing equipment and brought to approximately 1200 psi at about 38° C. and a flow rate of 68 slpm. The sonic probe was adjusted to 60%/o of total output power at a frequency of 20 kHz. The ethanol solution containing the docetaxel was pumped through the nozzle at a flow rate of 2 mL/minute for approximately 95 minutes). The precipitated docetaxel agglomerated particles and smaller docetaxel particles were then collected from the supercritical carbon dioxide as the mixture is pumped through the stainless steel mesh filter. The filter containing the particles of docetaxel was opened and the resulting product was collected from the filter.

As disclosed in U.S. Pat. No. 9,814,685, dissolution studies showed an increased dissolution rate in methanol/water media of paclitaxel and docetaxel particles made by the supercritical carbon dioxide methods described in U.S. Pat. No. 9,814,685 as compared to paclitaxel and docetaxel particles made by milling paclitaxel and docetaxel using a Deco-PBM-V-0.41 ball mill using a 5 mm ball size, at 600 RPM for 60 minutes at room temperature. The procedures used to determine the dissolution rates are as follows. For paclitaxel, approximately 50 mg of material were coated on approximately 1.5 grams of 1 mm glass beads by tumbling the material and beads in a vial for approximately 1 hour. Beads were transferred to a stainless steel mesh container and placed in the dissolution bath containing methanol/water 50/50 (v/v) media at 37° C., pH 7, and a USP Apparatus II (Paddle), operating at 75 rpm. At 10, 20, 30, 60, and 90 minutes, a 5 mL aliquot was removed, filtered through a 0.22 μm filter and analyzed on a UV/VIS spectrophotometer at 227 nm. Absorbance values of the samples were compared to those of standard solutions prepared in dissolution media to determine the amount of material dissolved. For docetaxel, approximately 50 mg of material was placed directly in the dissolution bath containing methanol/water 15/85 (v/v) media at 37° C., pH 7, and a USP Apparatus 11 (Paddle), operating at 75 rpm. At 5, 15, 30, 60, 120 and 225 minutes, a 5 mL aliquot was removed, filtered through a 0.22 μm filter, and analyzed on a UV/VIS spectrophotometer at 232 nm. Absorbance values of the samples were compared to those of standard solutions prepared in dissolution media to determine the amount of material dissolved. For paclitaxel, the dissolution rate was 47% dissolved in 30 minutes for the particles made by the supercritical carbon dioxide method versus 32% dissolved in 30 minutes for the particles made by milling. For docetaxel, the dissolution rate was 27% dissolved in 30 minutes for the particles made by the supercritical carbon dioxide method versus 9% dissolved in 30 minutes for the particles made by milling.

In some embodiments, the taxane particles have a SSA of at least 10, at least 12, at least 14, at least 16, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 m²/g. In one embodiment, the taxane particles have an SSA of between about 10 m²/g and about 50 m²/g. In some embodiments, the taxane particles have a bulk density between about 0.050 g/cm³ and about 0.20 g/cm³.

In further embodiments, the taxane particles have a SSA of:
(a) between 16 m²/g and 31 m²/g or between 32 m²/g and 40 m²/g;
(b) between 16 m²/g and 30 m²/g or between 32 m²/g and 40 m²/g,
(c) between 16 m²/g and 29 m²/g or between 32 m²/g and 40 m²/g;
(d) between 17 m²/g and 31 m²/g or between 32 m²/g and 40 m²/g;
(e) between 17 m²/g and 30 m²/g or between 32 m²/g and 40 m²/g;
(f) between 17 m²/g and 29 m²/g, or between 32 m²/g and 40 m²/g;
(g) between 16 m²/g and 31 m²/g or between 33 m²/g and 40 m²/g
(h) between 16 m²/g and 30 m²/g or between 33 m²/g and 40 m²/g;
(i) between 16 m²/g and 29 m²/g or between 33 m²/g and 40 m²/g;
(j) between 17 m²/g and 31 m²/g or between 33 m²/g and 40 m²/g;
(k) between 17 m²/g and 30 m²/g or between 33 m²/g and 40 m²/g,
(l) between 17 m²/g and 29 m²/g, or between 33 m²/g and 40 m²/g;
(m) between 16 m²/g and 31 m²/g, or ≥32 m²/g;
(h) between 17 m²/g and 31 m²/g, or ≥32 m²/g;
(i) between 16 m²/g and 30 m²/g, or ≥32 m²/g;
(j) between 17 m²/g and 30 m²/g, or ≥32 m²/g;
(k) between 16 m²/g and 29 m²/g, or ≥32 m²/g;
(l) between 17 m²/g and 29 m²/g, or ≥32 m²/g;
(m) between 16 m²/g and 31 m²/g, or ≥33 m²/g;
(n) between 17 m²/g and 31 m²/g, or ≥33 m²/g;
(o) between 16 m²/g and 30 m²/g, or ≥33 m²/g;
(p) between 17 m²/g and 30 m²/g, or ≥33 m²/g;
(q) between 16 m²/g and 29 m²/g, or ≥33 m²/g; or
(r) between 17 m²/g and 29 m²/g, or ≥33 m²/g.

In some embodiments, the taxane particles are non-agglomerated individual particles and are not clusters of multiple taxane particles that are bound together by interactive forces such as non-covalent interactions, van der Waal forces, hydrophilic or hydrophobic interactions, electrostatic interactions, Coulombic forces, interactions with a dispersion material, or interactions via functional groups. In some embodiments, the taxane particles are individual taxane particles that are formed by the agglomeration of smaller particles which fuse together forming the larger individual taxane particles, all of which occurs during the processing of the taxane particles.

In some embodiments, the taxane particles are paclitaxel particles and have an SSA of at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 m²/g. In other embodiments, the paclitaxel particles have an SSA of 18 m²/g to 50 m²/g, or 20 m²/g to 50 m²/g, or 22 m²/g to 50 m²/g, or 25 m²/g to 50 m²/g, or 26 m²/g to 50 m²/g, or 30 m²/g to 50 m²/g, or 35 m²/g to 50 m²/g, or 18 m²/g to 45 m²/g, or 20 m²/g to 45 m²/g, or 22 m²/g to 45 m²/g, or 25 m²/g to 45 m²/g, or 26 m²/g to 45 m²/g or 30 m²/g to 45 m²/g, or 35 m²/g to 45 m²/g, or 18 m²/g to 40 m²/g, or 20 m²/g to 40 m²/g, or 22 m²/g to 40 m²/g, or 25 m²/g to 40 m²/g, or 26 m²/g to 40 m²/g, or 30 m²/g to 40 m²/g, or 35 m²/g to 40 m²/g.

In some embodiments, the paclitaxel particles have a bulk density (not-tapped) of 0.05 g/cm³ to 0.15 g/cm³, or 0.05 g/cm³ to 0.20 g/cm³.

In some embodiments, the paclitaxel particles have a dissolution rate of at least 40% w/w dissolved in 30 minutes or less in a solution of 50% methanol/50% water (v/v) in a USP II paddle apparatus operating at 75 RPM, at 37° C., and at a pH of 7.

In some embodiments, the taxane particles are docetaxel particles and have an SSA of at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, or at least 42 m²/g. In other embodiments, the docetaxel particles have an SSA of 18 m²/g to 60 m²/g, or 22 m²/g to 60 m²/g, or 25 m²/g to 60 m²/g, or 30 m²/g to 60 m²/g, or 40 m²/g to 60 m²/g, or 18 m²/g to 50 m²/g, or 22 m²/g to 50 m²/g, or 25 m²/g to 50 m²/g, or 26 m²/g to 50 m²/g, or 30 m²/g to 50 m²/g, or 35 m²/g to 50 m²/g, or 40 m²/g to 50 m²/g.

In some embodiments, the docetaxel particles have a bulk density (not-tapped) of 0.05 g/cm³ to 0.15 g/cm³.

In some embodiments, the docetaxel particles have a dissolution rate of at least 20% w/w dissolved in 30 minutes or less in a solution of 15% methanol/85% water (v/v) in a USP II paddle apparatus operating at 75 RPM, at 37° C., and at a pH of 7.

The taxane particles can be packaged into any suitable container such as glass or plastic vials. A non-limiting example of a suitable container is a Type 1, USP, clear-glass vial closed with a bromobutyl rubber stopper and aluminum crimp seal. The taxane particles can be sterilized after the particles are in the container using sterilization methods known in the art such as gamma irradiation or autoclaving.

### Compositions

The compositions of the disclosure comprise taxane particles and are useful for treating kidney tumors direct injection of the compositions, i.e., intratumoral injection. The compositions can further comprise a carrier. The carrier can be a liquid (fluid) carrier, such as an aqueous carrier. Non-limiting examples of suitable aqueous carriers include water, such as Sterile Water for Injection USP; normal saline solution (0.9% sodium chloride solution), such as 0.9% Sodium Chloride for Injection USP; dextrose solution, such as 5% Dextrose for Injection USP; and Lactated Ringer's Solution for Injection USP. Non-aqueous based liquid carriers and other aqueous-based liquid carriers can be used. The carrier can be a pharmaceutically acceptable carrier, i.e., suitable for administration to a subject by injection or other routes of administration. The carrier can be any other type of liquid such as emulsions or flowable semi-solids. Non-limiting examples of flowable semisolids include gels and thermosetting gels. The composition can be a suspension, i.e., a suspension dosage form composition where the taxane particles are dispersed (suspended) within a continuous carrier/and or diluent. The taxane particles can be completely dispersed, partially dispersed and partially dissolved, but not completely dissolved in the carrier. In some embodiments, the composition is a suspension of taxane particles dispersed within a continuous carrier. In a preferred embodiment, the carrier is a pharmaceutically acceptable carrier. In preferred embodiments, the composition is sterile. In various embodiments, the composition comprises, consists essentially of, or consists of taxane particles and a liquid carrier, wherein the composition is a suspension of the taxane particles dispersed within the liquid carrier. In some embodiments, the composition consists essentially of or consists of taxane particles and a carrier, wherein the carrier is an aqueous carrier and wherein the composition is a suspension.

The composition of taxane particles and a carrier can be administered as-is. Optionally, the composition of taxane particles and a carrier can further comprise a suitable diluent to dilute the composition in order to achieve a desired concentration (dose) of taxane particles. In some embodiments, the carrier can serve as the diluent; stated another way, the amount of carrier in the composition provides the desired concentration of taxane particles in the composition and no further dilution is needed. A suitable diluent can be a fluid, such as an aqueous fluid. Non-limiting examples of suitable aqueous diluents include water, such as Sterile Water for Injection USP; normal saline solution (0.9% sodium chloride solution), such as 0.9% Sodium Chloride for Injection USP; dextrose solution, such as 5% Dextrose for Injection USP; and Lactated Ringer's Solution for Injection USP. Other liquid and aqueous-based diluents suitable for administration by injection can be used and can optionally include salts, buffering agents, and/or other excipients. In some embodiments, the diluent is sterile. The composition can be diluted with the diluent at a ratio to provide a desired concentration dosage of the taxane particles. For example, the volume ratio of composition to diluent might be in the range of 1:1-1:100 v/v or other suitable ratios. In some embodiments, the composition comprises taxane particles, a carrier, and a diluent, wherein the carrier and diluent form a mixture, and wherein the composition is a suspension of taxane particles dispersed in the carrier/diluent mixture. In some embodiments, the carrier/diluent mixture is a continuous phase and the taxane particles are a dispersed phase.

The composition, carrier, and/or diluent can further comprise functional ingredients such as buffers, salts, osmotic agents, surfactants, viscosity modifiers, rheology modifiers, suspending agents, pH adjusting agents such as alkalinizing agents or acidifying agents, tonicity adjusting agents, preservatives, antimicrobial agents including quaternary ammonium compounds such as benzalkonium chloride and benzethonium chloride, demulcents, antioxidants, antifoaming agents, alcohols such as ethanol, chelating agents, and/or colorants. For example, the composition can comprise taxane particles and a carrier comprising water, a salt, a surfactant, and optionally a buffer. In one embodiment, the carrier is an aqueous carrier and comprises a surfactant, wherein the concentration of the surfactant is 1% or less on a w/w or w/v basis; in other embodiments, the surfactant is less than 0.5%, less than 0.25%, less than 0.1%, or about 0.1%. In other embodiments, the aqueous carrier excludes the surfactants GELUCIRE® (polyethylene glycol glycerides composed of mono-, di- and triglycerides and mono- and diesters of polyethylene glycol) and/or CREMOPHOR® (polyethoxylated castor oil). In some embodiments, the composition or carrier excludes polymers, proteins (such as albumin), polyethoxylated castor oil, and/or polyethylene glycol glycerides composed of mono-, di- and triglycerides and mono- and diesters of polyethylene glycol.

The composition, carrier, and/or diluent can comprise one or more surfactants. Suitable surfactants include by way of example and without limitation polysorbates, lauryl sulfates, acetylated monoglycerides, diacetylated monoglycerides, and poloxamers, such as poloxamer 407. Polysorbates are polyoxyethylene sorbitan fatty acid esters which are a series of partial fatty acid esters of sorbitol and its anhydrides copolymerized with approximately 20, 5, or 4 moles of ethylene oxide for each mole of sorbitol and its anhydrides. Non-limiting examples of polysorbates are polysorbate 20, polysorbate 21, polysorbate 40, polysorbate 60, polysorbate 61, polysorbate 65, polysorbate 80, polysorbate 81, polysorbate 85, and polysorbate 120. Polysorbates containing approximately 20 moles of ethylene oxide are hydrophilic nonionic surfactants. Examples of polysorbates containing approximately 20 moles of ethylene oxide include polysorbate 20, polysorbate 40, polysorbate 60, polysorbate 65, polysorbate 80, polysorbate 85, and polysorbate 120. Polysorbates are available commercially from Croda under the tradename TWEEN™. The number designation of the polysorbate corresponds to the number designation of the TWEEN, e.g., polysorbate 20 is TWEEN 20, polysorbate 40 is TWEEN 40, polysorbate 60 is TWEEN 60, polysorbate 80 is TWEEN 80, etc. USP/NF grades of polysorbate include polysorbate 20 NF, polysorbate 40 NF, polysorbate 60 NF, and polysorbate 80 NF. Polysorbates are also available in PhEur grades (European Pharmacopoeia), BP grades, and JP grades. The term “polysorbate” is a non-proprietary name. The chemical name of polysorbate 20 is polyoxyethylene 20 sorbitan monolaurate. The chemical name of polysorbate 40 is polyoxyethylene 20 sorbitan monopalmitate. The chemical name of polysorbate 60 is polyoxyethylene 20 sorbitan monostearate. The chemical name of polysorbate 80 is polyoxyethylene 20 sorbitan monooleate. In some embodiments, the composition, carrier, and/or diluent can comprise mixtures of polysorbates. In some embodiments, the composition, carrier, and/or diluent comprises polysorbate 20, polysorbate 40, polysorbate 60, polysorbate 65, polysorbate 80, polysorbate 85, and/or polysorbate 120. In some embodiments, the composition, carrier, and/or diluent comprises polysorbate 20, polysorbate 40, polysorbate 60, and/or polysorbate 80. In one embodiment, the composition, carrier, and/or diluent comprises polysorbate 80.

In some embodiments, the composition, carrier, and/or diluent can comprise an alcohol, such as ethanol. The ethanol can be USP grade such as Alcohol USP or Dehydrated Alcohol (200 proof) USP. In some embodiments, the composition comprises taxane particles, a carrier, and optionally a diluent, wherein the carrier and/or diluent comprises water, ethanol, and a polysorbate. In some embodiments, the ethanol is present in the composition, carrier, and/or diluent at a concentration of about 0.1% w/v to about 10% w/v, or about 0.1% w/v to about 8% w/v, or about 2% w/v to about 8% w/v, or about 5% w/v to about 10% w/v, or about 8% w/v. In some embodiments, the ethanol is present in the composition at a concentration of about 0.1 w/v to about 4% w/v, or about 2% w/v to about 4% w/v, or about 3.2% w/v. In one embodiment, the composition is a suspension and the polysorbate is polysorbate 80. In other embodiments, the polysorbate or polysorbate 80 is present in the composition, carrier, and/or diluent at a concentration of between about 0.01% w/v and about 1.5% w/v. The inventors have surprisingly discovered that the recited very small amounts of polysorbate 80 reduce the surface tension at the interface of the taxane particles and the aqueous carrier (such as normal saline solution). These embodiments are typically formulated near the time of use of the composition. In some embodiments, the particles may be coated with the polysorbate or polysorbate 80. In other embodiments, the particles are not coated with the polysorbate or polysorbate 80. In various other embodiments, the polysorbate or polysorbate 80 is present in the composition, carrier, and/or diluent at a concentration of between: about 0.01% w/v and about 1% w/v, about 0.01% w/v and about 0.5% w/v, about 0.01% w/v and about 0.4% w/v, about 0.01% w/v and about 0.35% w/v, about 0.01% w/v and about 0.3% w/v, about 0.01% w/v and about 0.25% w/v, about 0.01% w/v and about 0.2% w/v, about 0.01% w/v and about 0.15% w/v, about 0.01% w/v and about 0.1% w/v, 0.02% w/v and about 1% w/v, about 0.02% w/v and about 0.5% w/v, about 0.02% w/v and about 0.4% w/v, about 0.02% w/v and about 0.35% w/v, about 0.02% w/v and about 0.3% w/v, about 0.02% w/v and about 0.25% w/v, about 0.02% w/v and about 0.2% w/v, about 0.02% w/v and about 0.15% w/v, about 0.02% w/v and about 0.1% w/v, about 0.05% w/v and about 1% w/v, about 0.05% w/v and about 0.5% w/v, about 0.05% w/v and about 0.4% w/v, about 0.05% w/v and about 0.35% w/v, about 0.05% w/v and about 0.3% w/v, about 0.05% w/v and about 0.25% w/v, about 0.05% w/v and about 0.2% w/v, about 0.05% w/v and about 0.15% w/v, about 0.05% w/v and about 0.1% w/v, about 0.1% w/v and about 1% w/v, about 0.1% w/v and about 0.5% w/v, about 0.1% w/v and about 0.4% w/v, about 0.1% w/v and about 0.35% w/v, about 0.1% w/v and about 0.3% w/v, about 0.1% w/v and about 0.25% w/v, about 0.1% w/v and about 0.2% w/v, about 0.1% w/v and about 0.15% w/v, about 0.2% w/v and about 1% w/v, about 0.2% w/v and about 0.5% w/v, about 0.2% w/v and about 0.4% w/v, about 0.2% w/v and about 0.35% w/v, about 0.2% w/v and about 0.3% w/v, about 0.2% w/v and about 0.25% w/v, about 0.3% w/v and about 1% w/v, about 0.3% w/v and about 0.5% w/v, about 0.3% w/v and about 0.4% w/v, or about 0.3% w/v and about 0.35% w/v; or about 0.01%, about 0.05%, about 0.1% w/v, about 0.15% w/v, about 0.16% w/v, about 0.2% w/v, about 0.25% w/v, about 0.3% w/v, about 0.35% w/v, about 0.4% w/v, about 0.45% w/v, about 0.5% w/v, or about 1% w/v.

The composition, carrier, and/or diluent can comprise one or more tonicity adjusting agents. Suitable tonicity adjusting agents include by way of example and without limitation, one or more inorganic salts, electrolytes, sodium chloride, potassium chloride, sodium phosphate, potassium phosphate, sodium, potassium sulfates, sodium and potassium bicarbonates and alkaline earth metal salts, such as alkaline earth metal inorganic salts, e.g., calcium salts, and magnesium salts, mannitol, dextrose, glycerin, propylene glycol, and mixtures thereof.

The composition, carrier, and/or diluent can comprise one or more buffering agents. Suitable buffering agents include by way of example and without limitation, dibasic sodium phosphate, monobasic sodium phosphate, citric acid, sodium citrate, tris(hydroxymethyl)aminomethane, bis(2-hydroxyethyl)iminotris-(hydroxymethyl)methane, and sodium hydrogen carbonate and others known to those of ordinary skill in the art. Buffers are commonly used to adjust the pH to a desirable range for intratumoral use.

The composition, carrier, and/or diluent can comprise one or more demulcents. A demulcent is an agent that forms a soothing film over a mucous membrane, such as the membranes lining the peritoneum and organs therein. A demulcent may relieve minor pain and inflammation and is sometimes referred to as a mucoprotective agent. Suitable demulcents include cellulose derivatives ranging from about 0.2 to about 2.5% such as carboxymethylcellulose sodium, hydroxyethyl cellulose, hydroxypropyl methylcellulose, and methylcellulose; gelatin at about 0.01%; polyols in about 0.05 to about 1%, also including about 0.05 to about 1%, such as glycerin, polyethylene glycol 300, polyethylene glycol 400, and propylene glycol; polyvinyl alcohol from about 0.1 to about 4%; povidone from about 0.1 to about 2%; and dextran 70 from about 0.1% when used with another polymeric demulcent described herein.

The composition, carrier, and/or diluent can comprise one or more alkalinizing agents to adjust the pH. As used herein, the term “alkalizing agent” is intended to mean a compound used to provide an alkaline medium. Such compounds include, by way of example and without limitation, ammonia solution, ammonium carbonate, potassium hydroxide, sodium carbonate, sodium bicarbonate, and sodium hydroxide and others known to those of ordinary skill in the art

The composition, carrier, and/or diluent can comprise one or more acidifying agents to adjust the pH. As used herein, the term “acidifying agent” is intended to mean a compound used to provide an acidic medium. Such compounds include, by way of example and without limitation, acetic acid, amino acid, citric acid, nitric acid, fumaric acid and other alpha hydroxy acids, hydrochloric acid, ascorbic acid, and nitric acid and others known to those of ordinary skill in the art.

The composition, carrier, and/or diluent can comprise one or more antifoaming agents. As used herein, the term “antifoaming agent” is intended to mean a compound or compounds that prevents or reduces the amount of foaming that forms on the surface of the fill composition. Suitable antifoaming agents include by way of example and without limitation, dimethicone, SIMETHICONE, octoxynol and others known to those of ordinary skill in the art.

The composition, carrier, and/or diluent can comprise one or more viscosity modifiers that increase or decrease the viscosity of the suspension. Suitable viscosity modifiers include methylcellulose, hydroxypropyl methycellulose, mannitol, polyvinylpyrrolidone, cross-linked acrylic acid polymers such as carbomer, and others known to those of ordinary skill in the art. The composition, carrier, and/or diluent can further comprise rheology modifiers to modify the flow characteristics of the composition to allow it to adequately flow through devices such as injection needles or tubes. Non-limiting examples of viscosity and rheology modifiers can be found in “Rheology Modifiers Handbook—Practical Use and Application” Braun, William Andrew Publishing, 2000.

The concentrations of taxane particles in the compositions can be at amounts effective for treatment of kidney tumors by direct injection of the compositions (intratumoral injection). In one embodiment, the concentration of the taxane particles in the composition is between about 0.1 mg/mL and about 100 mg/mL. In various further embodiments, the concentration of taxane particles in the composition is between: about 0.5 mg/mL and about 100 mg/mL, about 1 mg/mL and about 100 mg/mL, about 2 mg/mL and about 100 mg/mL, about 5 mg/mL and about 100 mg/mL, about 10 mg/mL and about 100 mg/mL, about 25 mg/mL and about 100 mg/mL, about 30 mg/mL and about 100 mg/mL, about 0.1 mg/mL and about 75 mg/mL, about 0.5 mg/mL and about 75 mg/mL, about 1 mg/mL and about 75 mg/mL, about 2 mg/mL and about 75 mg/mL, about 5 mg/mL and about 75 mg/mL, about 10 mg/mL and about 75 mg/mL, about 25 mg/mL and about 75 mg/mL, about 30 mg/mL and about 75 mg/mL, about 0.1 mg/mL and about 50 mg/mL, about 0.5 mg/mL and about 50 mg/mL, about 1 mg/mL and about 50 mg/mL, about 2 mg/mL and about 50 mg/mL, about 5 mg/mL and about 50 mg/mL, about 10 mg/mL and about 50 mg/mL, about 25 mg/mL and about 50 mg/mL, about 30 mg/mL and about 50 mg/mL, about 0.1 mg/mL and about 40 mg/mL, about 0.5 mg/mL and about 40 mg/mL, about 1 mg/mL and about 40 mg/mL, about 2 mg/mL and about 40 mg/mL, about 5 mg/mL and about 40 mg/mL, about 10 mg/mL and about 40 mg/mL, about 25 mg/mL and about 40 mg/mL, about 30 mg/mL and about 40 mg/mL, about 0.1 mg/mL and about 30 mg/mL, about 0.5 mg/mL and about 30 mg/mL, about 1 mg/mL and about 30 mg/mL, about 2 mg/mL and about 30 mg/mL, about 5 mg/mL and about 30 mg/mL, about 10 mg/mL and about 30 mg/mL, about 25 mg/mL and about 30 mg/mL, about 0.1 mg/mL and about 25 mg/mL, about 0.5 mg/mL and about 25 mg/mL, about 1 mg/mL and about 25 mg/mL, about 2 mg/mL and about 25 mg/mL, about 5 mg/mL and about 25 mg/mL, about 10 mg/mL and about 25 mg/mL, about 0.1 mg/mL and about 20 mg/mL, about 0.5 mg/mL and about 20 mg/mL, about 1 mg/mL and about 20 mg/mL, about 2 mg/mL and about 20 mg/mL, about 5 mg/mL and about 20 mg/mL, about 10 mg/mL and about 20 mg/mL, about 0.1 mg/mL and about 15 mg/mL, about 0.5 mg/mL and about 15 mg/mL, about 1 mg/mL and about 15 mg/mL, about 2 mg/mL and about 15 mg/mL, about 5 mg/mL and about 15 mg/mL, about 10 mg/mL and about 15 mg/mL, about 0.1 mg/mL and about 10 mg/mL, about 0.5 mg/mL and about 10 mg/mL, about 1 mg/mL and about 10 mg/mL, about 2 mg/mL and about 10 mg/mL, about 5 mg/mL and about 10 mg/mL, about 0.1 mg/mL and about 5 mg/mL, about 0.5 mg/mL and about 5 mg/mL, about 1 mg/mL and about 5 mg/mL, about 2 mg/mL and about 5 mg/mL, about 0.1 mg/mL and about 2 mg/mL, about 0.5 mg/mL and about 2 mg/mL, about 1 mg/mL and about 2 mg/mL, about 0.1 mg/mL and about 1 mg/mL, about 0.5 mg/mL and about 1 mg/mL, about 0.1 mg/mL and about 0.5 mg/mL, about 3 mg/mL and about 8 mg/mL, or about 4 mg/mL and about 6 mg/mL; or at least about 0.1, 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 61, 65, 70, 75, or 100 mg/mL; or about 0.1, 0.5, 1, 2, 3, 4, 5, 6, 7, 8, 9, 10, 11, 12, 13, 14, 15, 16, 17, 18, 19, 20, 25, 30, 35, 40, 41, 42, 43, 44, 45, 46, 47, 48, 49, 50, 55, 60, 61, 65, 70, 75, or 100 mg/mL. The taxane particles may be the sole therapeutic agent administered, or may be administered with other therapeutic agents.

In various embodiments, the composition comprises paclitaxel or docetaxel particles, a carrier, and a diluent, wherein the concentration of paclitaxel or docetaxel particles in the composition (including the carrier and diluent) is from about 0.1 mg/mL to about 40 mg/mL, or from about 1 mg/mL to about 30 mg/mL, or from about 1 mg/mL to about 20 mg/mL, or from about 1 mg/mL to about 15 mg/mL, or from about 1 mg/mL to about 10 mg/mL, or from about 1 mg/mL to about 8 mg/mL, or from about 1 mg/mL to about 4 mg/mL, or about 1 mg/mL, or about 2 mg/mL, or about 3 mg/mL, or about 4 mg/mL, or about 5 mg/mL, or about 6 mg/mL, or about 7 mg/mL, or about 8 mg/mL. In further embodiments, the carrier is an aqueous carrier which can be a saline solution, such as normal saline solution and the diluent is an aqueous diluent which can be a saline solution, such as normal saline solution. In further embodiments, the aqueous carrier comprises a polysorbate, such as polysorbate 80, and/or ethanol.

### Kits

The present disclosure also provides kits, comprising:
(a) a first vial comprising, consisting essentially of, or consisting of taxane particles having a mean particle size (number) of from 0.1 to 5 microns;
(b) a second vial comprising a pharmaceutically acceptable carrier; and
(c) instructions for reconstituting the taxane particles into a suspension useful for intratumoral injection into a kidney tumor by: combining the contents of the first vial and the second vial to form the suspension and optionally diluting the suspension with a diluent.

In some embodiments, the taxane particles are docetaxel or paclitaxel particles. The docetaxel or paclitaxel particles in the first vial can be in a powder form. The amount of docetaxel or paclitaxel particles in the first vial can be at any amount suitable for a desired dose level after reconstituting the particles into a suspension. In one embodiment, the taxane particles are docetaxel particles and the amount of docetaxel particles in the first vial is 100 mg. In another embodiment, the taxane particles are paclitaxel particle and the amount of paclitaxel particles in the first vial is 306 mg. The docetaxel or paclitaxel particles in the first vial can be the sole ingredient in the first vial. In some embodiments, the docetaxel or paclitaxel particles have a mean particle size (number) of from 0.1 microns to 1.5 microns. In other embodiments, the docetaxel or paclitaxel particles have a mean particle size (number) of from 0.4 microns to 1.2 microns. In some embodiments, the docetaxel or paclitaxel particles particles have a specific surface area (SSA) of at least 18 m²/g; and/or a bulk density (not-tapped) of 0.05 g/cm³ to 0.15 g/cm³. The pharmaceutically acceptable carrier can be an aqueous carrier such as normal saline solution. The carrier can further comprise a surfactant such as a polysorbate. In some embodiments, the polysorbate is polysorbate 80. In some embodiments, the polysorbate or polysorbate 80 is at a concentration of between about 0.01% w/v and about 1% w/v. In some embodiments, the amount of polysorbate 80 in the carrier in the second vial is about 1% w/v. In some embodiments, the carrier can further comprise an alcohol such as ethanol. In some embodiments, the amount of ethanol in the carrier in the second vial is about 8% w/v. The kits can include multiple vials of taxane particles and carrier solutions to allow for large volumes of reconstituted suspension. The kit can further comprise a diluent such as normal saline solution. The amount of diluent can be adjusted to provide a desired dose concentration and volume. When the suspension of docetaxel particles and a carrier containing a polysorbate and optionally ethanol is diluted with the diluent, excessive dissolution of the docetaxel particles is prevented.

Any suitable vial can be used in the kits. A non-limiting example of a suitable vial is a Type 1, USP, clear-glass vial closed with a bromobutyl rubber stopper and aluminum crimp seal. The volumes of the vials can vary depending on the amount of taxane particles, the volume of the carrier, and the volume of the final reconstituted suspension. The vials and their contents can be sterilized using sterilization methods known in the art such as gamma irradiation or autoclaving. In some embodiments, the contents of the vials are sterile. The kits can be configured for single-dose or multiple-dose administration.

A non-limiting exemplary procedure for preparing a suspension composition from a kit is as follows:

1. Using a syringe with a suitable gauge needle, add all or a portion of the carrier from the second vial into the first vial containing the anti-neoplastic particles.
2. Vigorously hand shake the first vial with inversions to make sure all the particles adhering to the interior of the vial and stopper are wetted.
3. Continue shaking for 1 minute and examine the suspension for any large clumps of particles.
4. Immediately after shaking, use a syringe with a suitable gauge needle to add a prescribed volume of a diluent to the first vial to dilute the suspension to a desired dose level, and hand shake the vial for another 1 minute. Periodically examine the suspension for any large visible clumps. If present, continue hand mixing until the suspension is properly dispersed.
5. After mixing, allow the suspension to sit undisturbed for at least 5 minutes to reduce entrapped air and foam.

The compositions, suspensions, and kits of the disclosure can include any embodiment or combination of embodiments described herein including any embodiments of the taxane particles, any embodiments of the carriers and diluents, any embodiments of the polysorbate or polysorbate 80 concentrations, and any embodiments of the ethanol concentrations. The compositions, suspensions, and kits can exclude polymers, proteins (such as albumin), polyethoxylated castor oil, and/or polyethylene glycol glycerides composed of mono-, di- and triglycerides and mono- and diesters of polyethylene glycol. The compositions and kits can further comprise other components as appropriate for given taxane particles.

### Methods of Administration/Treatment

The compositions comprising taxane particles described and disclosed herein can be used in methods for the treatment of kidney tumors by intratumoral injection administration of the composition. As used herein, the terms “intratumoral injection”, “intratumoral administration”, or “intratumoral injection administration” means that some or all of the composition, such as a suspension, is directly injected into a kidney tumor mass, and can include one or more injections at one or more injection sites in the tumor in a single administration of a single dose. As will be understood by those of skill in the art, such direct injection may include injection of some portion of the composition on the periphery of the kidney tumor (“peritumorally”), such as if the dose amount of composition or suspension thereof is too large to all be directly injected into the solid tumor mass. In one embodiment, the composition or suspension thereof is injected in its entirety into the kidney tumor mass. Intratumoral injection of compositions of the taxane particles into the tumor may be accomplished by any suitable means known by one of skill in the art. In non-limiting embodiments, the injection may be carried out via magnetic resonance imaging-transrectal ultrasound fusion (MR-TRUS) guidance (such as for injecting prostate tumors), or via endoscopic ultrasound-guided fine needle injection (EUS-FNI). Suitable intratumoral injection methods and compositions are disclosed in international patent application publication WO 2017/176628, herein incorporated by reference.

Multiple doses of the composition can be administered in two or more separate administrations. The two or more separate administrations can be administered at or at least 1, 2, 3, 4, 5, 6, 7, 8, 9, 10, 11, 12, or 14 days apart. In some embodiments, the two or more separate administrations are administered 2 to 12, 2-11, 2-10, 2-9, 2-8 2-7, 2-6, 2-5, 2-4, 2-3, 3-12, 3-11, 3-10, 3-9, 3-8, 3-7, 3-6, 3-5, 3-4, 4-12, 4-11, 4-10, 4-9, 4-8, 4-7, 4-6, 4-5, 5-12, 5-11, 5-10, 5-9, 5-8, 5-7, 5-6, 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, 11-12, 2, 3, 4, 5, 6, 7, 8, 9, 10, 11, or 12 weeks apart. In some embodiments, the composition is administered in 2-5, 2-4, 2-3, 3-5, 3-4, 2, 3, 4, 5, or more separate administrations. The two or more separate administrations can be administered once a week for at least two weeks. The two or more separate administrations can be administered twice a week for at least one week, wherein the two or more separate administrations are separated by at least one day. In some embodiments, the composition is administered in 1, 2, 3, 4, 5, 6, or more separate administrations. In some embodiments, the method results in elimination of the tumor. In other embodiments, the composition is administered in 7 or more separate administrations.

The kidney tumor can be malignant (cancerous) or benign (non-cancerous).

Treatment methods of the disclosure can include one or more tumors in one or both kidneys. Types of malignant tumors include but are not limited to renal cell carcinoma (RCC), clear cell renal cell carcinoma, papillary renal cell carcinoma, chromophobe renal cell carcinoma, collecting duct RCC, multilocular cystic RCC, medullary carcinoma, mucinous tubular and spindle cell carcinoma, neuroblastoma-associated RCC, unclassified RCCs, transitional cell carcinoma aka urothelial carcinoma, Wilms tumor (nephroblastoma), and renal sarcoma. Types of benign kidney tumors include but are not limited to renal adenoma, oncocytoma, and angiomyolipoma.

### EXAMPLES

The present disclosure will be described in greater detail by way of specific examples. The following examples are offered for illustrative purposes only, and are not intended to limit the disclosure in any manner. Those of skill in the art will readily recognize a variety of noncritical parameters, which can be changed or modified to yield essentially the same results.

### Example 1. Production of Paclitaxel Particles and Docetaxel Particles

### Materials and Methods

Raw paclitaxel and docetaxel were purchased from Phyton Biotech (British Columbia, Canada), lot number FP2-15004 and DT7-14025, respectively. Both were characterized in their raw form. The milling of both drugs was accomplished using a Deco-PBM-V-0.41 mill (Deco). The milling conditions for both compounds were as follows:
Ball size=5 mm
RPM=600
Processing time=60 min
Room temperature.

### Preparation of Paclitaxel Particles

A solution of 65 mg/mL of paclitaxel was prepared in acetone. A BETE MicroWhirl® fog nozzle (BETE Fog Nozzle, Inc) and a sonic probe (Qsonica, model number Q700) were positioned in the crystallization chamber approximately 8 mm apart. A stainless steel mesh filter with approximately 100 nm holes was attached to the crystallization chamber to collect the precipitated paclitaxel nanoparticles. The supercritical carbon dioxide was placed in the crystallization chamber of the manufacturing equipment and brought to approximately 1200 psi at about 38° C. and a flow rate of 24 kg/hour. The sonic probe was adjusted to 60% of total output power at a frequency of 20 kHz. The acetone solution containing the paclitaxel was pumped through the nozzle at a flow rate of 4.5 mL/minute for approximately 36 hours. Paclitaxel nanoparticles produced had an average number-weighted mean size of 0.81 μm with an average standard deviation of 0.74 μm over three separate runs.

### Preparation of Docetaxel Particles

A solution of 79.32 mg/mL of docetaxel was prepared in ethanol. The nozzle and a sonic probe were positioned in the pressurizable chamber approximately 9 mm apart. A stainless steel mesh filter with approximately 100 nm holes was attached to the pressurizable chamber to collect the precipitated docetaxel nanoparticles. The supercritical carbon dioxide was placed in the pressurizable chamber of the manufacturing equipment and brought to approximately 1200 psi at about 38° C. and a flow rate of 68 slpm. The sonic probe was adjusted to 60% of total output power at a frequency of 20 kHz. The ethanol solution containing the docetaxel was pumped through the nozzle at a flow rate of 2 mL/minute for approximately 95 minutes). The precipitated docetaxel agglomerated particles and smaller docetaxel particles were then collected from the supercritical carbon dioxide as the mixture is pumped through the stainless steel mesh filter. The filter containing the nanoparticles of docetaxel was opened and the resulting product was collected from the filter.

Docetaxel nanoparticles produced had an average number-weighted mean size of 0.82 μm with an average standard deviation of 0.66 μm over three separate ethanol runs.

### Particle Size Analysis

Particle size was analyzed by both light obscuration and laser diffraction methods. A Particle Sizing Systems AccuSizer 780 SIS system was used for the light obscuration method and Shimadzu SALD-7101 was used for the laser diffraction method. Paclitaxel nanoparticles were analyzed using 0.10% (w/v) sodium dodecyl sulfate (SDS) in water as the dispersant. Docetaxel nanoparticles were analyzed using Isopar G as the dispersant.

Paclitaxel suspensions were prepared by adding approximately 7 mL of filtered dispersant to a glass vial containing approximately 4 mg of paclitaxel particles. The vials were vortexed for approximately 10 seconds and then sonicated in a sonic bath approximately 1 minute. If the sample was already suspended, 1:1 solution of paclitaxel suspension to 0.1% SDS solution was made, vortexed for 10 seconds, and sonicated in the sonic bath for 1 minute.

Docetaxel suspensions were prepared by adding approximately 7 mL of filtered dispersant to a plastic vial containing approximately 4 mg of docetaxel particles. The vial was vortexed for approximately 10 seconds and then sonicated in a sonic bath for approximately 2 minutes. This suspension was used for laser diffraction analysis. Unused suspension was poured into a 125 mL particle-free plastic bottle, which was then filled to approximately 100 mL with filtered dispersant. The suspension was vortex for approximately 10 seconds and then sonicated in the sonic bath for approximately 2 minutes. This diluted suspension was used for light obscuration analysis.

A background test was first performed prior to analyzing particles on the AccuSizer 780 SIS. A new particle-free plastic bottle was filled with blank suspension solution by pumping from a reservoir, using a peristaltic pump, through a 0.22 μm Millipore filter and into the bottle. A background analysis was run to ensure the particle/mL count was below 100 particles/mL. A small amount of paclitaxel suspension, 5-100 μL, depending upon concentration of solution, was pipetted into the plastic bottle in place from the background test and was filled with ˜100 mL dispersant and the analysis was started. Counts were monitored and paclitaxel solution added to reach and/or maintain 6000-8000 particle counts/mL during the entire analysis. Once the analysis was completed, the background data was removed and any measurement with less than four counts was removed.

To analyze particles on SALD-7101 using a batch cell, the analysis was started by choosing Manual Measurement. The refractive index was set as 1.5 to 1.7. The batch cell was filled with filtered dispersant just past the etched line. The blank measurement was ran. A small amount of API (paclitaxel or docetaxel) suspension was pipetted, generally <1 mL, depending upon concentration of solution as low as 100 μL, into the batch cell as needed to achieve an acceptable absorbance between 0.15 and 0.2 absorbance units. The measurements were executed, and the resulting graph with the highest level of confidence was selected; background was automatically accounted for.

### BET Analysis

A known mass between 200 and 300 mg of the analyte was added to a 30 mL sample tube. The loaded tube was then mounted to a Porous Materials Inc. SORPTOMETER®, model BET-202A. The automated test was then carried out using the BETWIN® software package and the surface area of each sample was subsequently calculated.

### Bulk Density Analyte

Paclitaxel or docetaxel particle preparations were added to a 10 mL tared graduated cylinder through a plastic weigh funnel at room temperature. The mass of the drug was measured to a nearest 0.1 mg, the volume was determined to the nearest 0.1 mL and the density calculated.

### Dissolution Studies

### Paclitaxel

Approximately 50 mg of material (i.e.: raw paclitaxel, milled paclitaxel, or paclitaxel particles) were coated on approximately 1.5 grams of 1 mm glass beads by tumbling the material and beads in a vial for approximately 1 hour. Beads were transferred to a stainless steel mesh container and placed in the dissolution bath containing methanol/water 50/50 (v/v) media at 37° C., pH 7, and a USP Apparatus II (Paddle), operating at 75 rpm. At 10, 20, 30, 60, and 90 minutes, a 5 mL aliquot was removed, filtered through a 0.22 μm filter and analyzed on a U (V/V) is spectrophotometer at 227 nm. Absorbance values of the samples were compared to those of standard solutions prepared in dissolution media to determine the amount of material dissolved.

### Docetaxel

Approximately 50 mg of material (i.e.: raw docetaxel, milled docetaxel, or docetaxel particles) was placed directly in the dissolution bath containing methanol/water 15/85 (v/v) media at 37° C., pH 7, and a USP Apparatus II (Paddle), operating at 75 rpm. At 5, 15, 30, 60, 120 and 225 minutes, a 5 mL aliquot was removed, filtered through a 0.22 μm filter, and analyzed on a UV/VIS spectrophotometer at 232 nm. Absorbance values of the samples were compared to those of standard solutions prepared in dissolution media to determine the amount of material dissolved.

### Results

The BET surface area of particles produced using the above protocol and variations thereof (i.e.: modifying nozzles, filters, sonic energy sources, flow rates, etc.) ranged between 22 and 39 m²/g. By comparison, the BET surface area of raw paclitaxel was measured at 7.25 m²/g, while paclitaxel particles made according to the methods of U.S. Pat. Nos. 5,833,891 and 5,874,029 ranged from 11.3 to 15.58 m²/g. Exemplary particle sizes produced using the methods of the disclosure are shown in Table 1.

TABLE 1

| | | |
|---|---|---|
| Surface | Mean Size | St Dev |
| area | μm | μm |

| | m²/g | Number | Volume | Number | Volume |
|---|---|---|---|---|---|
| 1 | 38.52 | 0.848 | 1.600 | 0.667 | 0.587 |
| 2 | 33.82 | 0.754 | 0.988 | 0.536 | 0.486 |
| 3 | 35.90 | 0.777 | 1.259 | 0.483 | 0.554 |
| 4 | 31.70 | 0.736 | 0.953 | 0.470 | 0.466 |
| 5 | 32.59 | 0.675 | 0.843 | 0.290 | 0.381 |
| 6 | 38.22 | 0.666 | 0.649 | 0.344 | 0.325 |
| 7 | 30.02 | 0.670 | 0.588 | 0.339 | 0.315 |
| 8 | 31.16 | 0.672 | 0.862 | 0.217 | 0.459 |
| 9 | 23.90 | 0.857 | 1.560 | 0.494 | 0.541 |
| 10 | 22.27 | 0.857 | 1.560 | 0.494 | 0.541 |
| 11 | 26.19 | 0.861 | 1.561 | 0.465 | 0.546 |

Comparative studies on bulk density, SSA, and dissolution rates (carried out as noted above) for raw drug, milled drug particles, and drug particles produced by the methods of the present disclosure are provided in Table 2 and Table 3 below. The full dissolution time course for the paclitaxel and docetaxel materials are provided in Table 4 and Table 5, respectively.

TABLE 2
Compound: Paclitaxel

| | Raw | Particles | | | |
|---|---|---|---|---|---|
| Characteristic | Material | Batch 1 | Batch 2 | Mean | Milled |
| Number Mean (um) | 1.16 | 0.83 | 0.67 | 0.75 | 0.89 |
| Volume Mean (um) | 1.29 | 1.42 | 0.57 | 1.00 | 1.35 |
| Bulk Density (g/cm³) | 0.26 | 0.060 | 0.11 | 0.085 | 0.31 |
| Surface Area (m²/g) | 10.4 | 35.6 | 39.8 | 37.7 | 15.0 |
| Dissolution (30 min) | 18% | 42% | 52% | 47% | 32% |

TABLE 3
Compound: Docetaxel

| | Raw | Particles | | | |
|---|---|---|---|---|---|
| Characteristic | Material | Batch 1 | Batch II | Mean | Milled |
| Number Mean (um) | 1.58 | 0.92 | 0.80 | 0.86 | 1.11 |
| Volume Mean (um) | 5.05 | 4.88 | 4.03 | 4.46 | 3.73 |
| Bulk Density (g/cm³) | 0.24 | 0.062 | 0.096 | 0.079 | 0.44 |
| Surface Area (m²/g) | 15.9 | 43.0 | 45.4 | 44.2 | 15.2 |
| Dissolution (30 min) | 11% | 27% | 27% | 27% | 9% |

TABLE 4
Paclitaxel Dissolution time course

| Timepoint | Paclitaxel Raw | Paclitaxel | Milled |
|---|---|---|---|
| (minutes) | Material | Particles | Paclitaxel |
| 0 | 0.0% | 0.0% | 0.0% |
| 10 | 14.0% | 40.2% | 23.0% |
| 20 | 17.8% | 47.6% | 30.0% |
| 30 | 18.4% | 51.9% | 32.3% |
| 60 | 23.9% | 58.3% | 38.6% |
| 90 | 28.6% | 62.9% | 43.5% |

TABLE 5
Docetaxel Dissolution time course

| Timepoint | Docetaxel Raw | Docetaxel | Milled |
|---|---|---|---|
| (minutes) | Material | Particles | Docetaxel |
| 0 | 0.0% | 0.0% | 0.0% |
| 5 | 3.2% | 12.1% | 3.2% |
| 15 | 6.9% | 21.7% | 5.9% |
| 30 | 11.2% | 27.2% | 9.3% |
| 60 | 16.4% | 32.9% | 12.2% |
| 120 | 22.4% | 38.9% | 13.6% |
| 225 | 26.8% | 43.1% | 16.0% |

### Example 2. Drug Efficacy Study in Rat Xenograft Model of Human Renal Cell Adenocarcinoma

A non-GLP study was conducted to determine the drug efficacy of nPac (nanoparticle paclitaxel) suspension and nDoce (nanoparticle docetaxel) suspension administered by intratumoral injections in a rat xenograft model of human renal cell adenocarcinoma.

### Objectives

The objective of this study was to investigate the potential efficacy of nPac (nanoparticle paclitaxel) and nDoce (nanoparticle docetaxel), administered by intratumoral (IT) injections over a period of time in the Sprague-Dawley Rag2; Il2rg null (SRG®) rat xenograft model of human renal cell adenocarcinoma (786-O cell line) (ATCC® CRL-1932™). Five to seven weeks old SRG rats were inoculated with 5 million 786-O cells in Cultrex® subcutaneously to develop tumor xenograft. Once the tumor volume reached 150-300 mm³, the rats were enrolled on a rolling basis into treatment groups consisting of the test articles (administered IT); positive controls (paclitaxel and docetaxel; administered intravenous (IV)) and a vehicle control (administered IT), then monitored for the tumor growth or regression.

### Cell Culture

Cell lines: 786-O cell line (ATCC® CRL-1932™). Cells were stored in liquid nitrogen. Upon thawing, cells were cultured at 37° C., 5% CO2. After cells were prepared for transplant, they were maintained on ice until injection.

Cell culture conditions: Cells were cultured in RPMI 1640 (Gibco #410491-01)+10% FBS on tissue-culture treated flasks at 37° C., 5% CO2. Cells were expanded for 2-3 weeks prior to inoculation. Cell thawing, culturing and passaging was carried by ATCC (www.atcc.org/Products/All/CRL-1932.aspx)

Cell Inoculation: 5×10⁶ cells per rat; subcutaneous left hind flank, dorsal side.

Inoculation vehicle: 50% Cultrex BME type 3 (Trevigen #3632-001-02; a type of basement membrane matrix like Matrigel® formulated for in vivo tumor growth) 50% Media in a total volume of 0.5 ml. Cell suspension mixed 1:1 with IOmg/mL Cultrex for a final concentration of 5 mg/mL Cultrex. Final inoculation volume is 500 ul.

Preparation of Test Articles (nPac and nDoce Suspension)

Drug: nPac (nanoparticle paclitaxel powder, approximately 98% paclitaxel with a mean particle size (number) of 0.878 microns, a SSA of 26.7 m²/g, and a bulk density (not tapped) of 0.0763 g/cm³ used in this example) 306 mg in a 60 mL vial; and nDoce (nanoparticle docetaxel powder, approximately 99% docetaxel with a mean particle size (number) of 1.078 microns, a SSA of 37.2 m²/g, and a bulk density (not tapped) of 0.0723 g/cm³ used in this example) 100 mg in a 60 mL vial.

For nPac Suspension (Final concentration: 20 mg/mL nPac and 0.32% Polysorbate 80 in normal saline solution—Final volume: 15.3 mL per vial):

Using a sterile syringe with a sterile 18-gauge needle or larger, added 5.0 mL of a sterile 1% polysorbate 80 reconstitution solution into the 60 ml nPac powder vial (containing 306 mg nPac powder).

Vigorously hand shook the vial with inversions to make sure all the particles adhering to the interior of the vial and stopper are wetted.

Continued shaking for 1 minute and examined the suspension for any large clumps of particles.

Immediately after shaking, used a sterile syringe with a sterile 18-gauge needle or larger to add 10.3 mL of a normal saline solution (0.9% sodium chloride solution for injection) to the vial and hand shook the vial for another 1 minute. Periodically examined the suspension for any large visible clumps. If present, continued hand mixing until the suspension was properly dispersed.

After mixing, allowed the suspension to sit undisturbed for at least 5 minutes to reduce entrapped air and foam.

For nDoce Suspension

(Final concentration: 20 mg/mL nDoce, 0.20% Polysorbate 80, and 1.6% ethanol in normal saline solution—Final volume: 5 mL per vial):

Using a sterile syringe with a sterile 18-gauge needle or larger, added 1 mL of a sterile 1% polysorbate 80/8% ethanol reconstitution solution into the 60 ml nDoce powder vial (containing 100 mg nDoce powder).

Vigorously hand shook the vial with inversions to make sure all the particles adhering to the interior of the vial and stopper are wetted.

Continued shaking for 1 minute and examined the suspension for any large clumps of particles.

Immediately after shaking, used a sterile syringe with a sterile 18-gauge needle or larger to add 4 mL of normal saline solution (0.9% sodium chloride for injection) to the vial and hand shook the vial for another 1 minute. Periodically examined the suspension for any large visible clumps. If present, continued hand mixing until the suspension was properly dispersed.

After mixing, allowed the suspension to sit undisturbed for at least 5 minutes to reduce entrapped air and foam.

Intratumoral (IT) Vehicle

(Final concentration: 0.2% Polysorbate 80 and 1.6% ethanol in normal saline solution): Each 1 mL of a 1% Polysorbate/8% ethanol reconstitution solution was diluted with 4 mL of normal saline solution (0.9% sodium chloride solution for injection).

### Preparation of Positive Controls Formulation

Drug: Docetaxel: CAS 114977-28-5, and Paclitaxel: CAS 33069-62-4. Purity >97%

For Docetaxel Solution: Made a 20 mg/mL solution of docetaxel in 50% ethanol:50% Polysorbate 80. Vortexed to mix. Added normal saline solution to dilute to a 3 mg/mL solution of docetaxel.

For Paclitaxel Solution: Used bulk paclitaxel to make 6 mg/mL formulation in 50%

ethanol: 50% Cremophor EL. Vortexed as needed to mix. Added normal saline solution to dilute to a 3 mg/mL solution of paclitaxel. Vortex to mix.

### Test System

Species/Strain: Rat (Rattus norvegicus)/Rag2^{-/-}; 112 rg^{-/-} on Sprague Dawley background (SRG®).

Number of Animals/Approximate Age and Weight: Sixty healthy rats (30 males and 30 females) were assigned for this study and used for xenograft development. At least 54 tumor-bearing animals in total were enrolled for treatment (27 males and 27 females) as they reached the required tumor volume. These animals were inoculated with 786-O cells in staggered batches on the same day, pending animal availability. Animals were approximately 5-7 weeks of age at the onset of the study. Approximate weight was 150-275 g. Animals were enrolled in the treatment groups on a rolling basis when the tumor size reached 150-300 mm³.

### Organization of Treatment Groups, Dosage Levels and Treatment Regimen

Table 6 below presents the study group arrangement.

TABLE 6

| | | Dose route, | Dosage | Dose | Dose | Number |
|---|---|---|---|---|---|---|
| | | Dose | level | concentration | volume | of |
| Group | Treatment | Schedule | (mg/kg/day | (mg/ml) | (ml/kg) | rats* |
| 1 | Vehicle | IT, QW × 3 | 0 | N/A | 1 | 6 |
| 2 | Paclitaxel | IV, QW × 3 | 5 | 3 | 1.67 | 6 |
| 3 | nPac | IT, QW × 1 | 20 | 20 | 1 | 6 |
| 4 | nPac | IT, QW × 2 | 20 | 20 | 1 | 6 |
| 5 | nPac | IT, QW × 3 | 20 | 20 | 1 | 6 |
| 6 | Docetaxel | IV, QW × 3 | 2.5-5 | 3 | 0.835-1.67 | 6 |
| 7 | nDoce | IT, QW × 1 | 20 | 20 | 1 | 6 |
| 8 | nDoce | IT, QW × 2 | 20 | 20 | 1 | 6 |
| 9 | nDoce | IT, QW × 3 | 20 | 20 | 1 | 6 |

*3 males and 3 females were allocated per group.
** IT doses were administered as a maximum of 8 equal volume injections placed evenly across the tumor site.

### Treatment Regimen:

All rats that developed tumors that reached 150-300 mm³ in volume were enrolled in treatment. All treatment commenced after 7 days post inoculation when tumors were >150 mm³.

Groups 3, 4 and 5 rats received nPac and groups 7, 8 and 9 rats received nDoce. Groups 3 and 7 received IT injections only on staging day (first day of treatment), groups 4 and 8 received IT injections on staging day and 7 days post initiation of treatment, and groups 5 and 9 received IT injections on staging day, 7 and 14 days post initiation of treatment. Positive control test articles (paclitaxel and docetaxel) were administered intravenously by tail vein injection on staging day, 7 and 14 days post-initiation of treatment to Groups 2 (paclitaxel) and 6 (docetaxel) rats. The vehicle control was administered by IT injection on staging day, 7 and 14 days post-initiation of treatment to group 1 animals.

### Methods of Administration:

The test articles and the vehicle were administered by IT injections or IV injections depending on the dosing group, with sterile needles and syringes. All IV injections were administered using a 27G needle and each animal was injected with a fresh syringe.

IT injections were distributed across the tumor in up to 8 injections when the tumor was intact and 3 injections in case of an ulcerating tumor. The number of IT injections per tumor during all dosing days were recorded in the raw data.

The dose volume was 1 mL/kg for the vehicle, nPac and nDoce and 1.67 mL/kg for paclitaxel and docetaxel. For group 6, the dosage of Docetaxel was changed to 2.5 mL/kg and the dose volume was decreased to 0.835 mL/kg following observed toxicity on day 7. At the time of dose administration, nPac and nDoce vials were inverted gently 5-10 times immediately prior to dose removal to ensure uniformity of the suspension.

Using a sterile syringe with a sterile 18-gauge* needle or larger bore, inverted the vial and inserted the needle into the septum of the inverted vial. Withdrew just over the amount of suspension needed, removed the needle from the vial and adjusted to the desired volume. Recapped the needle. *Note: for IT injections, a 27G needle was used for administration.

IT injections were administered across the tumor in a Z pattern (across top, diagonal through, then across bottom) and reversed each following dosing occasion(s). The injections were administered with the needle bevel facing down to minimize leakage of the TA post injection. The skin was also pulled slightly back prior to needle entry and during the injection to also minimize TA leakage post injection. Efforts were made to ensure IT injection administration patterns are consistent across all animals and dosing days.

nPac was used within 1 hour and nDoce within 24 hours of reconstitution. The positive controls and docetaxel were maintained at room temperature and used within 8 hours of formulation while paclitaxel was kept in warm water after reconstitution and used within 20 minutes.

### Observations:

Individual Body Weights: Three times weekly (M, W, F) starting at the time of inoculation.

Individual Tumor Volumes: Animals were palpated daily starting the day after tumor inoculation. Tumor length and width were measured with digital calipers and recorded starting when tumor volume reached 50 mm³, at which point tumors were measured three times weekly (M, W, F) and at the time of necropsy. Tumor volume (mm³) was calculated as =(L×W²)/2 where ‘L’ is the largest diameter.

Tumor Imaging: Photographs of all tumors were taken on staging day prior to commencement of treatment and 7, 14, 21, 28, 35, and 42 days post initiation of treatment. Additional tumor photographs were also taken at the time of necropsy of all rats including animals reaching end-point before study termination. All photographs will be taken with the animal in an anterior-posterior orientation with a photo-tag that states the animal I.D., study day and date.

Blood Sample Collection for Analysis: 200-250 ul of blood was collected from the tail or jugular vein of all treated animals at study termination, i.e. 50 days post initiation of treatment.

Scheduled Necropsy: All animals were scheduled for necropsy 50 days post the initiation of the treatment. Day 0 was day of tumor inoculation.

### Anatomic Pathology:

Macroscopic Examination: A necropsy was conducted on all animals dying spontaneously, euthanized in extremis or at the scheduled necropsy after 50 days post initiation of treatment. Animals euthanized in extremis or at study termination were euthanized by CO2 inhalation. Necropsy included examination of the external surface, all orifices and the thoracic, abdominal and pelvic cavities, including viscera. At the time of necropsy, a final body weight and body condition score was collected.

Tissue Collection: Primary Tumor (Inoculation site)—A final tumor measurement was taken prior to excision. Tumors were weighed after excision. Approximately ½ of each tumor (based on visual assessment) was flash frozen in 2-methylbutane on dry ice, the tumor piece was weighed when possible before it is flash frozen. The remaining was fixed in 10% neutral buffered formalin. Tumors were also collected from animals not reaching enrollment volume. Secondary Tumors—Any organ with visible tumors were collected and fixed in 10% neutral buffered formalin. Formalin fixed tissues were stored at room temperature. Frozen tissues were stored at −80° C. All tissue was stored for up to 3 months. Pictures of all tumors; primary and secondary if present, were taken.

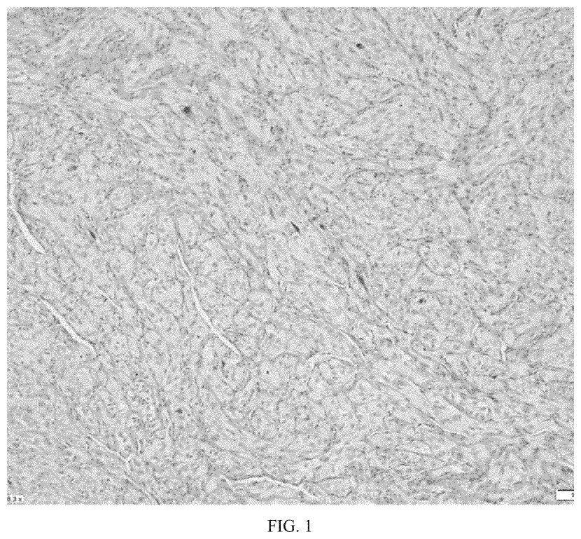

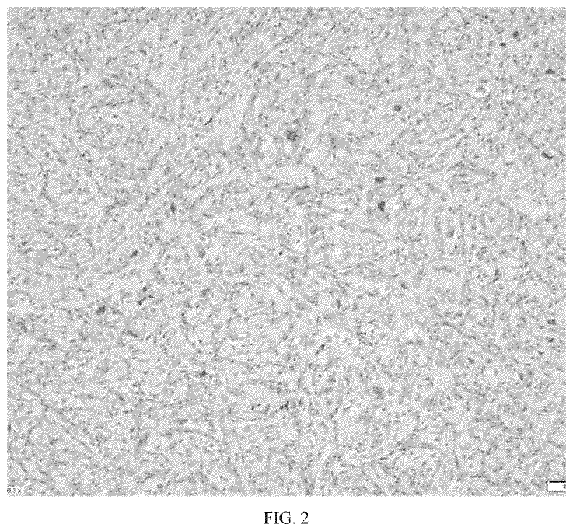

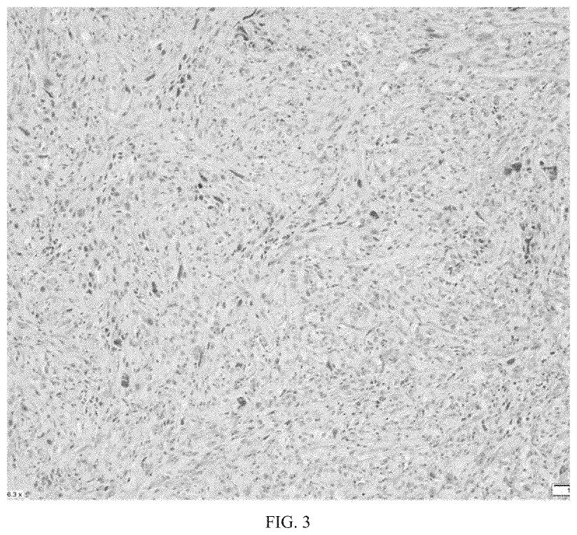

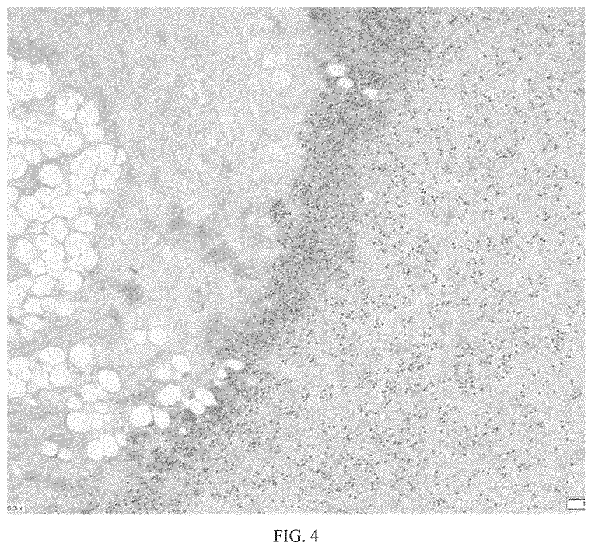

Microscopic Examination: Tissues fixed with 10% NBF were embedded in paraffin. Each tumor was cut into 2-3 pieces and embedded and sectioned together. For each tumor, 3 slides were prepared and stained with H&E. Photomicrographs of preliminary histology slides from female rats for Non-Treated, Vehicle Control (IT), Docetaxel (IV), and nDoce (IT) are shown in FIG. 1, FIG. 2, FIG. 3, and FIG. 4, respectively.

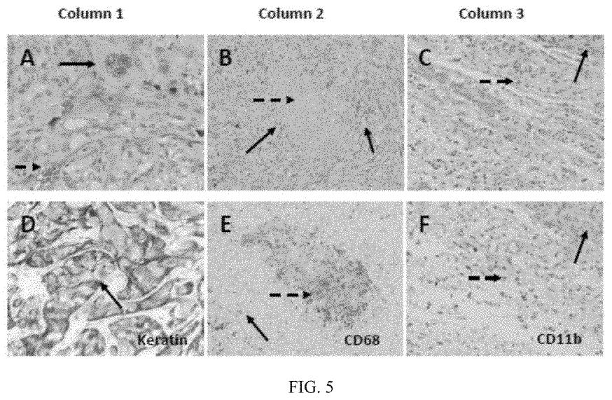

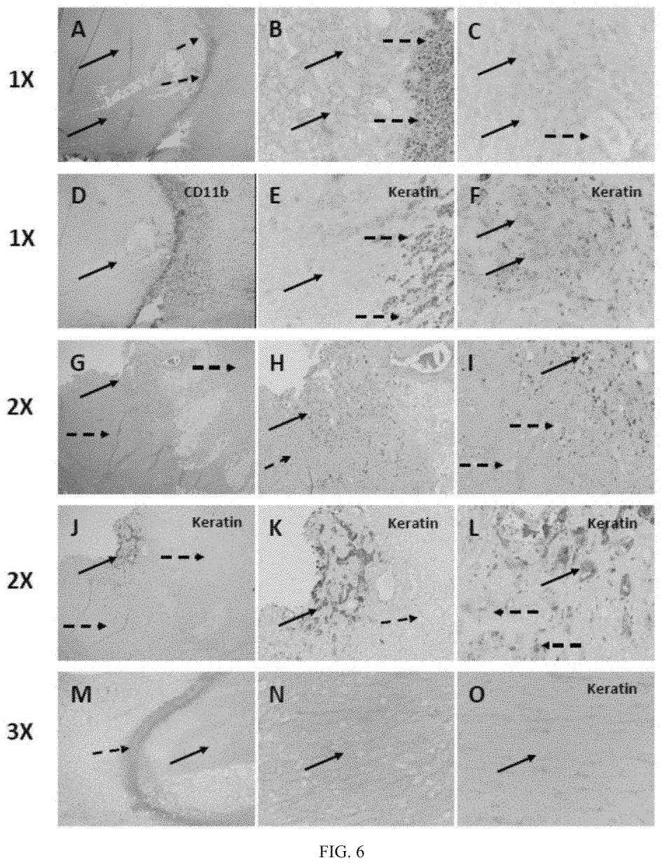

Additional H&E and Immunohistochemical (IHC) evaluations were conducted on formalin-fixed tissue from animals from the Docetaxel group and are shown in FIGS. 5 and 6.

### Histology Overview of Photomicrographs in FIG. 2, FIG. 3, and FIG. 4.

Vehicle Control (IT), FIG. 2: The photomicrograph shows “packets” of multi-/bi-nucleate tumor cells surrounded by extracellular matrix.

Docetaxel (IV), FIG. 3: The photomicrograph shows morphologically similar “packets” of viable renal cell carcinoma seen in the vehicle control: no difference.

nDoce (IT), FIG. 4: The photomicrograph shows a band of mononuclear cells representing a robust immune response to the tumor cells. Some dead tumor or dying tumor is present characterized as cellular “ghosts” (shown left of the mononuclear immune cell band). To the right of the mononuclear cell band are “ghosts” covered by a “sprinkling” of mononuclear immune cells.

### Additional H&E and Immunohistochemical (IHC) Evaluation of the Docetaxel Groups

Observations: FIG. 5 Control Cases. Top row: H&E stained sections. Bottom row: Immunohistochemical staining.

### Column 1:

(A) Renal cell carcinoma composed of closely apposed cohesive clusters and cords of large tumor cells with pleomorphic nuclei and visible nucleoli. Note the minimal intervening stroma that contains scattered small blood vessels (dashed arrow bottom left). Note multinucleated carcinoma cell at top of image (solid arrow). (D) Keratin (AE1/AE3) immunostain performed on the same tumor shown in A. This demonstrates sensitive and specific labeling of carcinoma cells with pancytokeratin (solid arrow).

### Column 2:

(B) Focal area of tumor cell necrosis composed of uniformly homogenous amorphous eosinophilic material (dashed arrow). Note the discrete nature of this focus with sharp demarcation from the surrounding viable carcinoma cells (solid arrows). This was the typical appearance of necrosis in the control groups. This was present in central areas of the tumor and occupied less than 5% of the tumor area. (E) CD68 stain (macrophage marker) highlighting the same area shown in image B. This shows limited numbers of macrophages in the viable carcinoma (solid arrow) and markedly increased macrophages in the focus of necrosis (dashed arrow). The latter finding illustrates the characteristic macrophage function of necrotic debris phagocytosis.

### Column 3:

(C) Limited numbers of small lymphocytes in the peritumoral surrounding non-neoplastic stroma (dashed arrow). Note carcinoma in top right corner (solid arrow). In the control groups, there were typically very few lymphocytes within the tumor itself and the peritumoral soft tissue generally contained a mild lymphoid infiltrate. (F) Corresponding focus to that seen in C, stained with CDIlb, showing positive staining in lymphoid cells (dashed arrow). Note carcinoma in top right corner (solid arrow).

Remarks: The two control cases demonstrated similar findings at the morphologic and immunohistochemical level. Both contained a dense nodule of invasive carcinoma that was sharply demarcated from the surrounding normal stromal tissue without a discrete well-formed fibrous capsule. Within the tumor nodule, the carcinoma cells were arranged into small organized clusters and cords of tumor cells and these were closely packed together with a minimal amount of intervening stoma that contained compressed small blood vessels (FIG. 5—Slide A). The tumor cells were large with pleomorphic nuclei that had vesicular chromatin and prominent eosinophilic nucleoli that were clearly visible at 100× magnification (10× eyepiece and 10× objective lens). The nuclei included rounded and spindled forms and scattered multinucleated giant tumor cells were present (FIG. 5—Slide A). The tumor cells had an abundant amount of lightly eosinophilic and clear cytoplasm and they showed increased mitotic activity (13 mitoses per 10 high power fields [400×hpf]). Scattered discrete foci of coagulative tumor cell necrosis were present and these were more frequent within central portions of the tumor nodule (FIG. 5—Slide B). The foci of necrosis consisted of homogenous eosinophilic necrotic debris that was relatively well demarcated from surrounding viable tumor cells. The foci of necrosis occupied less than 5% of the tumor cell area. Immunohistochemical staining for pancytokeratin (AE1/AE3) highlighted the tumor cells and displayed cytoplasmic and membranous localization (FIG. 5—Slide D). The keratin labeling was strong, sensitive and specific, with sharp demarcation between positively stained tumor cells and negatively stained surrounding non-carcinomatous tissue. There was no overt tumor regression noted in either of the two control group animals. There was no significant lymphoid infiltrate within the tumor. The surrounding stroma contained a patchy mild lymphoid infiltrate composed of scattered small lymphocytes that were mainly arranged as single cells (FIG. 5—Slide C). Immunohistochemical staining for CD11b (marker of NK cells and histiocytes) highlighted the mild immune cell infiltrate in the surrounding non-neoplastic stroma (FIG. 5.—Slide F); however, there was no significant lymphoid component within the tumor. Immunohistochemical staining for CD68 (marker of macrophages) highlighted a mild macrophage infiltrate within and around the tumor with increased density of staining within the foci of tumor necrosis, consistent with increased macrophages in areas containing increased cellular debris (FIG. 5.—Slide E).

Observations: FIG. 6. Intratumoral nDoce cases (representative images from all groups included: 1 cycle, 2 cycles and 3 cycles).

### Top Row:

One cycle nDoce (1×) (case 750-258). (A) Low power H&E staining showing extensive geographic tumor cell necrosis consisting of homogeneous eosinophilic staining of non-viable necrotic material (solid arrows). Note the central vertical line of demarcation consisting of a dense band of necrotic debris and admixed immune cells (dashed arrows) (B) High power view of line of demarcation. Note the dense collection of immune cells and admixed debris (dashed arrows at right). On the left of the image there is extensive necrotic material with no viable tumor cells (solid arrows) (C) High power view of the central portion of necrosis corresponding to the left half of image A. Solid arrows point to ghost outlines of necrotic tumor cells. The dashed arrow highlights a degenerating small blood vessel.

### Second Row:

One cycle nDoce (1×) (case 750-258). Each image corresponds to the H&E image above it. (D) CD11b immunostain of area seen in image A. This highlights the dense collection of immune cells in the central band of necrotic debris and immune cell infiltrate This stain also highlights immune cell response in the surrounding tissue at right but there is a lesser degree of inflammation in the central area of tumor necrosis at left. (E) Keratin stain showing the same area as seen in B. This shows complete absence of staining, thus adding strong immunohistochemical support for the interpretation of no residual viable carcinoma in this area. (F) Keratin stain from central area of necrosis shown in image C. This shows keratin labeling of degenerating keratin filaments in the necrotic ghost cell outlines (solid arrows) which supports the hypothesis that viable carcinoma subsequently underwent complete regression and necrosis; however, there are no residual viable tumor cells present in this area (lack of viable nuclei best appreciated in H&E image above).

### Third Row:

Two cycles nDoce (2×) (case 748-827). (G) H&E staining showing a 0.9 mm residual focus of viable carcinoma (solid arrow) surrounded by extensive necrotic material (dashed arrows). (H) Same focus of carcinoma at higher power showing viable tumor cells with retained nuclei (solid arrow). Note the progressive loss of viable tumor cells toward the lower left corner (dashed arrow) (I) Higher power of same focus illustrating the leading edge of the viable tumor (solid arrow) and the adjacent zone of tumor cell death. Here, remnants of tumor cells in progressive stages of cell death are evidenced by progressive loss of nuclei and loss of discrete cytoplasmic membrane outlines (dashed arrows).

### Fourth Row:

Two cycles nDoce (2×) (case 748-827). Each image corresponds to the H&E image above it. (J) Low power view of keratin stain with the focus of residual viable carcinoma in top left of image (solid arrow). Surrounding this focus is a lack of keratin staining (dashed arrows), exhibiting the extent of the necrotic material. (K) Higher power view of the same keratin-stained tumor showing viable nucleated carcinoma cells that label strongly with keratin antibody (solid arrow) and surrounding necrotic tissue that is negative for keratin staining (dashed arrow). (L) Keratin stain of the same area, illustrating progressive transition from viable nucleated keratin-positive carcinoma cells in top right (solid arrows) to tumor cells in varying stages of necrosis towards bottom left corner (dashed arrows). The latter include anuclear ghost outlines of tumor cells that show keratin labeling of residual degenerating tumor cell keratin intermediate filaments; however, these cells are non-viable. This supports the impression that the necrotic material surrounding the viable carcinoma previously contained viable carcinoma that subsequently died following therapy.

### Fifth Row:

Three cycles nDoce (3×) (case 748-822). (M) Low power H&E stained section showing dense amorphous necrosis on the right (solid arrow) that is demarcated from surrounding zone of degenerating fibrofatty tissue on the left by a band of necrotic debris and admixed immune cells (dashed arrow). (N) High power view of necrotic area showing no viable nucleated carcinoma cells (solid arrow). (O) Keratin stained section of same area in image N, showing complete absence of staining (solid arrow), thus further supporting an absence of residual carcinoma in this area following therapy.

Remarks:

Intratumoral nDoce 1 Cycle:

Two of the three animals in this group contained residual viable invasive carcinoma. When measured on the H/E stained slide this was significantly smaller in size (up to 5 mm in maximum cross-sectional dimension on the slide) compared to the control, IT vehicle and IV docetaxel groups (range of 9-15 mm with most of these being closer to 15 mm in maximum cross-sectional dimension on the slide). Where present, the morphology of the tumor cells in these two IT nDoce cases was essentially identical to that seen in the above-mentioned non-IT docetaxel groups. Both IT nDoce cases did not have sufficient a non-viable tumor or non-neoplastic stroma for evaluation of surrounding necrosis although one of these did have a focal peripheral rim of necrosis that occupied <5% of the submitted tissue. Similar to the control groups, there was only a mild immune cell infiltrate associated with these tumors in the surrounding non-neoplastic stromal tissue (where evaluable) and this was highlighted by a CD11b immunostain. The third animal in this group showed no viable residual invasive carcinoma and extensive geographic tumor cell coagulative necrosis. Extensive areas of necrosis blended with surrounding stromal fibrous, fatty and skeletal muscle tissue. In areas there was a line of demarcation between the amorphous necrosis and adjacent degenerating fibrofatty tissue which this consisted of a dense band of necrotic debris and admixed immune cells (FIG. 6—Slide A and B). No diagnostic viable tumor cells were noted on H/E stained section examination (FIG. 6—Slide B); however, in the central portion of the amorphous necrotic material there was a small area where ghost outlines of nuclear necrotic tumor cells were noted (FIG. 6—Slide C). This was also highlighted on the keratin-stained section where the keratin antibody labeled degenerating keratin filaments in the necrotic cell outlines (FIG. 6—Slide F). In addition, very focally within the degenerating and necrotic fibrofatty tissue, the keratin stained section of this animal showed focal cytoplasmic labeling that appeared consistent with histiocytic engulfment of degenerating keratin intermediate filaments. Of importance, the keratin stain did not show discrete cytoplasmic membrane labeling of viable carcinoma cells and it did not show any cohesive collections of keratin-labeled diagnostic viable tumor cells. In some areas there were abundant granular blue material that coalesced into small homogenous structures focally that were suggestive of dystrophic calcification. This granular material was difficult to definitively identify, and the differential diagnosis included granular necrotic debris and calcium, degenerating skeletal muscle fibers and nanoparticles. Immunohistochemical staining for CD11b in the animal with complete tumor regression highlighted by a moderate macrophage infiltrate in the non-neoplastic tissue and the CD11b stain also highlighted the zone of debris and admixed inflammation (FIG. 6—Slide D). Immunohistochemical staining for CD68 (marker of histiocytes) highlighted a moderate macrophage infiltrate.

Intratumoral nDoce 2 Cycles:

Two of the three animals (750-254 and 748-827) in this group contained residual viable invasive carcinoma. When measured on the H&E stained slide this was significantly smaller in size (3 mm and 0.9 mm in maximum cross-sectional dimension on the slide respectively) compared to the control, IT vehicle and IV docetaxel groups (range of 9-15 mm with most of these being closer to 15 mm in maximum cross-sectional dimension on the slide). In both IT nDoce cases with residual carcinoma, there was extensive geographic tumor cell necrosis surrounding the small foci of residual viable invasive carcinoma (FIG. 6—Slides G, H and I). Higher power examination of H&E stained and keratin stained sections from the smaller of these residual tumors showed a progressive transition from viable carcinoma cells to necrotic carcinoma cells with the latter being identified by labeling of their residual degenerating keratin intermediate filaments with the pancytokeratin immunostain (FIG. 6—Slides I and L). In both animals with residual carcinoma, immunohistochemical staining for CD11b highlighted a moderate immune cell infiltrate in the necrotic tissue. Immunohistochemical staining for CD68 (marker of histiocytes) highlighted a moderate macrophage infiltrate within the necrotic areas in both cases. The third case (748-826) in this group showed extensive geographic tumor cell coagulative necrosis with no residual viable invasive carcinoma noted on H&E or keratin-stained sections. Immunohistochemical staining for CD11b highlighted a patchy moderate immune cell infiltrate. Immunohistochemical staining for CD68 (marker of macrophages) highlighted a patchy moderate macrophage infiltrate.

Intratumoral nDoce 3 Cycles:

Both cases in this group (748-797 and 748-822) showed extensive geographic tumor cell coagulative necrosis with no residual viable invasive carcinoma noted on H&E or keratin-stained sections (FIG. 6—Slides M-O). Immunohistochemical staining for CD11b highlighted a moderate and marked immune cell infiltrate in the necrotic tissue in the two animals respectively. Immunohistochemical staining for CD68 (marker of histiocytes) highlighted a mild and marked macrophage infiltrate within the necrotic areas in these two cases, respectively.

Note: Animals in nDoce treatment groups had tumors with white “calcified” areas, likely resulting from nanoparticle deposits that remained within the tumor.

Additional Observations: (no figures)

IT nDoce Vehicle Group:

The two intratumoral vehicle cases demonstrated similar findings at the morphologic and immunohistochemical level and both essentially had an identical morphologic and immunohistochemical appearance to that seen in the control group.

### IV Docetaxel:

The two intratumoral IV docetaxel cases demonstrated similar findings at the morphologic and immunohistochemical level and both essentially had an identical morphologic and immunohistochemical appearance to that seen in the control and IT vehicle groups.

### Tumor Volume Results for Paclitaxel Group and Docetaxel Group:

Animals were weighed, and tumor length and width were measured with digital calipers three times weekly for 58 days and at the time of necropsy. Tumor volume (V) was calculated as follows: V (mm³)=((L*W²))/2

where L is the largest diameter and W is the width (in mm) of the tumor. Study Log® was employed for statistical analysis of tumor volume and body weight.

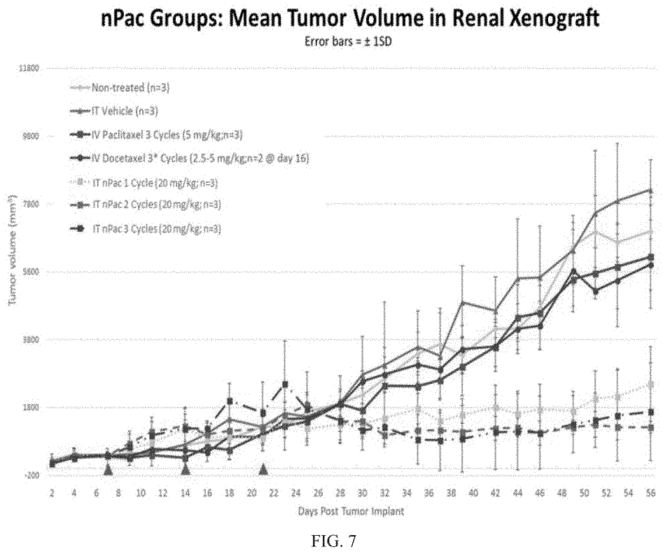

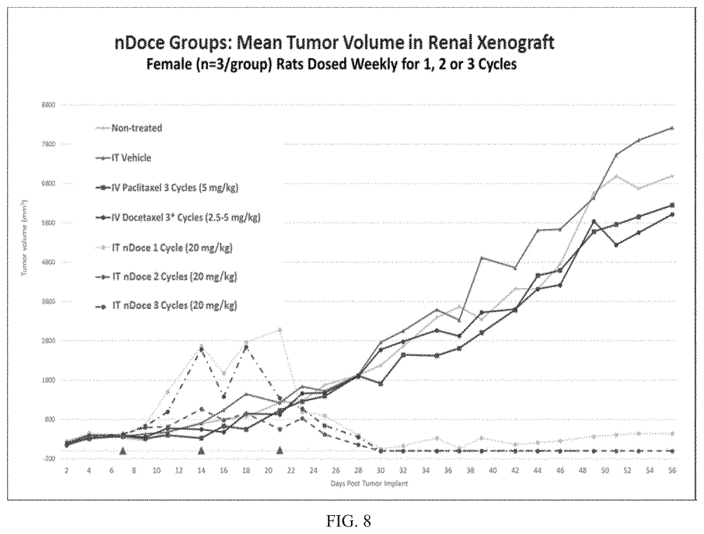

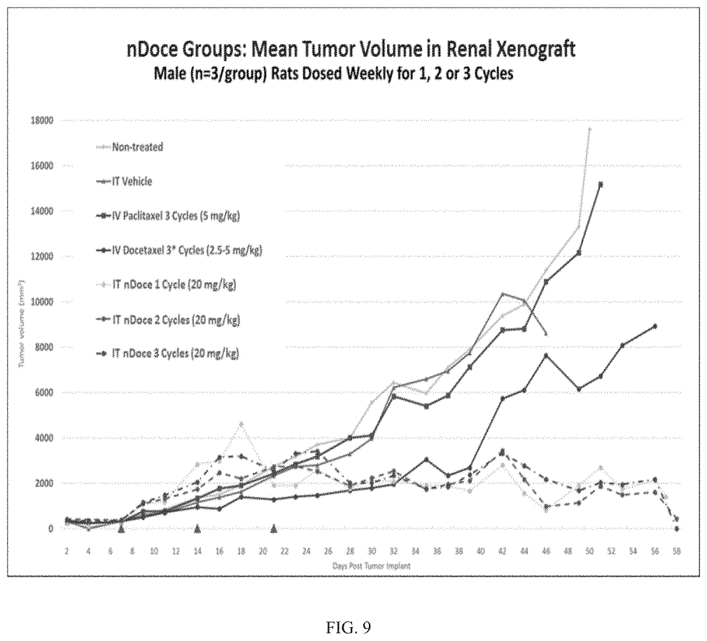

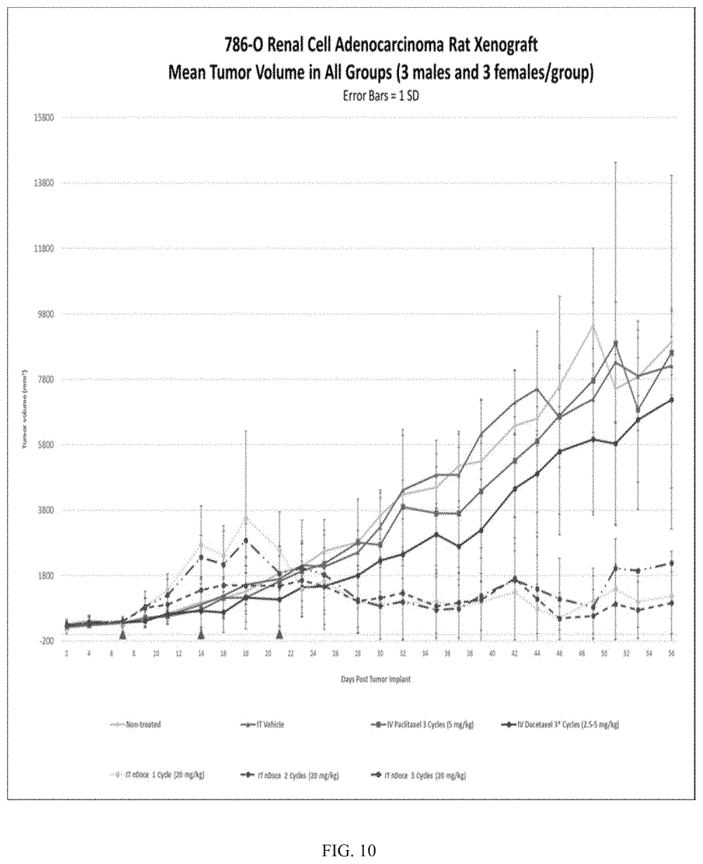

The mean tumor volume results of female rats for the Paclitaxel groups are shown in FIG. 7. Mean tumor volume results of female rats for the Docetaxel groups are shown in FIG. 8. Mean tumor volume results of male rats for the Docetaxel groups are shown in FIG. 9. Mean tumor volume results of female and male rats for the Docetaxel groups are shown in FIG. 10. As can be seen in the figures, IT nPac and IT nDoce both effectively treated the tumors.

Regarding the tumor volume results for the Docetaxel groups, the first measurable tumors for both males and females were observed at 2 days post-inoculation.

Non-treated and vehicle control-treated tumors continued to grow throughout treatment, with final volumes in female rats ranging from 5656 mm³ to less than 10,000 mm³. IV docetaxel treatment resulted in partial tumor growth inhibition compared to vehicle control.

nDoce delivered IT was the most efficacious treatment compared to vehicle and all other treatments. In most animals, the tumors treated with one, two or three cycles of IT nDoce appeared to have completely regressed with only necrotic tissue remaining at the original tumor site.

Upon necropsy, animals in nDoce treatment groups had tumors with white “calcified” areas, likely resulting from nanoparticle deposits that remained within the tumor.

### Docetaxel Group Results:

Docetaxel Concentration in Tissue: Tumor tissue concentrations of docetaxel were determined by LC-MS/MS analysis using its deuterated analogue docetaxel-d₉ as the internal standard. Using a method previously developed by Frontage, concentrations of docetaxel were obtained from calibration curves constructed by plotting the peak area ratios (analyte to internal standard) versus analyte concentration using linear regression with a weighting of 1/x². The nominal concentration range was 1.00-2,000 ng/g for docetaxel in tumor tissue. A calibration curve, prepared in rat control tumor tissue homogenate, was analyzed at the beginning and the end of each analytical run. Two sets of quality control (QC) samples were prepared at four concentration levels (low, mid-1, mid-2 and high) and were used to ensure reliability of the assay.

Thirty-eight days following the last of three weekly cycles of IV docetaxel (5-2.5 mg/kg), one of four animals evaluated had a detectable (LOQ=1.00 ng/g) docetaxel level of 21.8 ng/g. All three animals in the nDoce QWX1 group had detectable docetaxel levels ranging from 659 ng/g to 1.4×10⁵ ng/g 51 days post-treatment. Two animals from the nDoce QWX2 group were evaluated and had levels of 2.49 and 5.26 μg/g 44 days post-treatment. As there was no tumor available for analysis in the nDoce QWX3 group, no analysis was performed.

Animals: Throughout the treatment period, animals across all groups displayed relatively normal weight gain compared to non-treated animals and vehicle control with a few exceptions. One animal that received nDoce QWX1 had weight loss at treatment day 9. Despite supplementation she continued to lose weight and was subsequently euthanized on treatment day 16 due to reaching weight loss endpoints. One animal that received nDoce QWX3 lost a significant amount of weight, reaching endpoints at treatment day 39 despite supplementation.

Other observations include ulceration and apparent peripheral neuropathy. All animals that received nDoce exhibited ulcerations or lesions on the surface of the tumor. These lesions were described as “scabs”, areas of dry, rigid tissue. In most cases the wounds remained intact. A single animal that received nDoce QWX3 showed hindlimb weakness and limited mobility on day 35 post-treatment. With intervention, the weakness stabilized enough for the animal to remain in the study. However, the animal was euthanized on day 49 due to ulcerations that covered >50% of the tumor surface.

The ranges of sizes (the maximum cross-sectional dimension of the viable carcinoma as measured in millimeters on the slide) of the residual tumors in the six groups are shown in Table 7.

TABLE 7

| | | No viable | | | | |
|---|---|---|---|---|---|---|
| Group | # | tumor | <1 mm | 1-5 mm | 6-10 mm | >10 mm |
| Control | 2 | | | | | 2 |
| IT vehicle | 2 | | | | 1 | 1 |
| IV docetaxel | 2 | | | | | 2 |
| IT nDoce 1 | 3 | 1 | | 2 | | |
| IT nDoce 2 | 3 | 1 | 1 | 1 | | |
| IT nDoce 3 | 2 | 2 | | | | |

A condensation of the data in Table 7 which directly compares the size of the residual carcinoma nodules in the three non-nDoce groups (6 animals in total) with the three nDoce groups (8 animals in total) is shown in Table 8.

TABLE 8

| | | No viable | | | | |
|---|---|---|---|---|---|---|
| Group | # | tumor | <1 mm | 1-5 mm | 6-10 mm | >10 mm |
| non-nDoce | 6 | | | | 1 | 5 |
| IT nDoce | 8 | 4 | 1 | 3 | | |

Five of the six non-nDoce animals, including both IV docetaxel animals, had residual viable carcinoma nodules that measured greater than 10 mm, and most of these were closer to 15 mm. The remaining non-nDoce animal had viable carcinoma measuring 9 mm in maximum dimension. By contrast, half ( 4/8) of the animals treated with IT nDoce had no residual viable carcinoma on the slide to measure. All the remaining 4 animals in the IT nDoce group that had residual viable carcinoma had a viable carcinoma nodule that measured 5 mm or less in maximum dimension on the slide. This included one case where the tumor measured 0.9 mm, and this was not evident when the tumor was measured grossly prior to microscopic examination.

A comparison of the three IT nDoce groups with respect to percentage of cases with no residual invasive carcinoma and the size of residual viable carcinoma nodules is shown in Table 9.

TABLE 9

| | | | | | | % of cases |
|---|---|---|---|---|---|---|
| | | No | | | | with no |
| | | viable | | | Size of viable | residual |
| Group | # | tumor | <1 mm | 1-5 mm | nodules (mm) | carcinoma |
| IT Nano 1 | 3 | 1 | | 2 | 4, 5 | 33% |
| IT Nano 2 | 3 | 1 | 1 | 1 | 0.9, 3 | 33% |
| IT Nano 3 | 2 | 2 | | | N/A | 100% |

IT nDoce 1 and 2 cycle groups both had ⅓ of cases with no residual viable carcinoma while the IT nDoce 3 cycle group had 2/2 of cases with no residual viable invasive carcinoma. Amongst the cases with residual viable carcinoma, progressive increase in the number of cycles of IT nDoce was associated with a decrease in the size of the residual viable carcinoma nodule. Specifically, the residual viable carcinoma nodule measured 4 mm and 5 mm in the IT nDoce 1 cycle group and in the IT nDoce 2 cycle group the nodules measured 0.9 mm and 3 mm. There was no residual viable carcinoma to measure in the two cases in the IT nDoce 3 cycle group.

A percentage of tissue showing necrosis is shown in Table 10.

TABLE 10

| Groups | # | 100% | >90% | 50-90% | 5-50% | <5% |
|---|---|---|---|---|---|---|
| Control | 2 | | | | | 2 |
| IT vehicle | 2 | | | | | 2 |
| IV Doce | 2 | | | | | 2 |
| IT Nano 1 | 3 | 1 | | | | 2* |
| IT Nano 2 | 3 | 1 | 1 | 1 | | |
| IT Nano 3 | 2 | 2 | | | | |

All six animals in the non-nDoce group showed <5% necrosis. This consisted of focal small discrete foci of necrosis in the tumor that were small, occupying <5% of the tumor area, and they were within central portions of the tumor nodule, suggesting that these may be secondary to hypoxemia due to tumor outgrowing its blood supply. Four of the eight nDoce animals showed complete necrosis of tumor. Two of the four nDoce animals with residual carcinoma showed extensive necrosis in the surrounding tissue (>50% of tissue). *The two remaining nDoce animals with residual carcinoma did not have sufficient surrounding tissue for definitive assessment of necrosis although one of these did contain a focal rim of necrosis that represented <5% of the submitted tissue area.

The lymphohistiocytic infiltrate density based on assessment of H/E and immunohistochemical staining with CD11b, graded semi quantitatively is shown in Table 11.

TABLE 11

| Groups | # | Mild | Moderate | Marked |
|---|---|---|---|---|
| Control | 2 | 2 | | |
| IT vehicle | 2 | 2 | | |
| IV Doce | 2 | 2 | | |
| IT Nano 1 | 3 | 2 | 1 | |
| IT Nano 2 | 3 | | 3 | |
| IT Nano 3 | 2 | | 1 | 1 |

All six animals in the non-nDoce groups contained a mild immune cell infiltrate and this was present in the peritumoral non-neoplastic stroma without any significant immune cell infiltrate within the tumor. By contrast, 7 of the 8 animals in the nDoce groups contained a moderate immune cell infiltrate while the remaining animal had a marked immune cell infiltrate. This correlated with the increased amount of necrosis in the IT nDoce-treated animals.

### Discussion of Docetaxel Group Results:

A review was conducted on the morphologic and immunohistochemical features of a subset of 14 female rats from the renal cell carcinoma study aimed to assess the efficacy of intratumoral nDoce (the total study contained 30 animals). The current subset of 14 animals included two control animals, two animals given intratumoral vehicle, two animals treated with intravenous docetaxel (3 cycles) and eight animals treated with intratumoral nDoce. The nDoce group was separated into three groups based on the number of administered cycles: group 1 (1 cycle; 3 animals), group 2 (2 cycles; 3 animals), and group 3 (3 cycles; 2 animals).

The main feature that differed amongst the various groups was the presence and degree of tumor regression. In all animals in the intratumoral nDoce groups, tumor regression was prominent, while in all animals in the other groups, tumor regression was absent.

All six animals in the non-nDoce group (i.e. control, IT vehicle and IV docetaxel groups) had residual viable tumor. This consisted of a dense nodule of invasive carcinoma that was sharply demarcated from the surrounding normal stromal tissue. The carcinoma cells were closely packed together and while there were scattered discrete foci of coagulative tumor cell necrosis present, these were small in size, overall occupied <5% of the tumor area in each of the six animals, and were within central portions of the tumor nodule. These observations suggest that these areas of necrosis may be secondary to hypoxemia due to tumor outgrowing its blood supply (Table 10). Keratin staining showed strong, sensitive and specific staining of tumor cells. The maximum dimension of the viable tumor nodule, as measured on the stained slides, ranged from 9-15 mm in these six animals and in many this was closer to 15 mm (Tables 7 and 8). This tumor size on the slide corresponded to the tumor measurement taken at the time of gross dissection.

By contrast, four of the eight animals treated with intratumoral nDoce had no residual viable carcinoma as determined by assessment of H&E and keratin-stained sections (complete response). Of the remaining four animals, the residual viable tumor, as measured on the stained slide, was markedly smaller than that seen in the non-nDoce group (Tables 7 and 8). Specifically, the size of the residual viable tumor nodules in these four animals treated with IT nDoce ranged from 0.9 mm to 5 mm in maximum dimension (Table 9). In three of these animals, the tumor size measured on the slide correlated with the tumor size measurement taken at the time of gross dissection. In the remaining animal with a 0.9 mm focus of invasive carcinoma, this was present amongst extensive necrosis and was not evident at the time of gross dissection.

In six of the eight nDoce animals, there was extensive tumor cell coagulative necrosis that extended into adjacent necrotic skeletal muscle and fibrous tissue in some animals. In addition, focally within the necrotic areas there was keratin-staining of necrotic, non-viable, ghost tumor cell outlines, consistent with labelling of degenerating keratin intermediate filaments from dead tumor cells. This further supported that these areas previously contained viable carcinoma that had completely responded to therapy. In the slides from the two remaining animals there was very limited surrounding tissue for assessment of necrosis although one of these did contain a focal peripheral rim of necrosis in one area.

Within the non-nDoce group there was a uniformly mild immune cell infiltrate, and this was seen primarily in the non-neoplastic tissue surrounding the tumor. There was no significant intratumoral immune cell infiltrate. By contrast, the intratumoral nDoce group included two cases with a mild immune cell infiltrate, five cases with a moderate immune cell infiltrate and a single case with a marked immune cell infiltrate within the necrotic areas (Table 11). Like the non-nDoce group, there was no significant intratumoral lymphoid infiltration.

In summary, this review was limited to 14 female animals out of a study that contained 30 female and 30 male animals; however, a striking difference in the type and degree of tumor response to therapy was noted when the intratumoral nDoce group was compared to the non-nDoce groups. None of the six non-nDoce group animals showed any overt evidence of tumor regression and all had residual viable carcinoma nodules that ranged in size from 9-15 mm as measured on the slide. However, all eight animals in the intratumoral nDoce group showed evidence of tumor response and extensive necrosis was noted in all six of the animals that had sufficient surrounding tissue for assessment. The tumor response included compete regression in half of this group ( 4/8), as demonstrated by lack of definitive residual viable carcinoma on examination of H&E and keratin-stained sections, while the remaining four animals contained a focal small residual viable carcinoma nodule, the largest of which measured 5 mm and the smallest of which measured 0.9 mm. In two of these four animals with residual carcinoma, there was sufficient surrounding tissue present on the slides for assessment and this showed extensive necrosis. Similarly, the degree of immune cell infiltrate in the non-nDoce group was mild while it ranged from mild to marked in the nDoce group suggesting an association with the degree of tumor response and resultant necrotic debris.

When the three IT nDoce groups were compared with each other, it was noted that as the animals received increasing cycles of intratumoral nDoce therapy they showed a greater degree of tumor response. In particular, of the 3 animals in the group receiving 1 cycle of IT nDoce, one of three animals showed complete response while the remaining two animals had residual nodules measuring 4 and 5 mm. Of the three animals in the group receiving 2 cycles of IT nDoce, one showed complete response while the remaining two animals had residual nodules measuring 0.9 and 3 mm. Finally, both animals in the group receiving 3 cycles of IT nDoce showed complete response to therapy (two of two evaluated) (Table 9).

In conclusion, all eight animals with renal cell carcinoma in this study that were treated with intratumoral nDoce exhibited a notable histological response which included a 50% rate of complete tumor regression as well as a marked decrease in residual tumor size in the remaining four animals. Associated extensive necrosis and increased immune response was noted in the nDoce groups and focal areas of keratin-labelling of anuclear, non-viable, ghost tumor cell outlines in the necrotic areas further supported that these areas previously contained viable carcinoma that had completely responded to therapy. By contrast, there was no such tumor regression in the non-NanoDoc® e-treated groups. Furthermore, increasing cycles of intratumoral nDoce from 1 to 3 cycles resulted in a progressively greater degree of tumor regression and a progressively higher rate of complete regression within the IT nDoce cohort.

### Example 3. Phase 1/2 Trial Evaluating the Safety and Tolerability of the Intratumoral Injection of a Suspension of Nanoparticulate Docetaxel (abbreviated herein as “nDoce”) in Subjects with Localized Renal Cell Carcinoma

Summary: In this open-label, dose rising, Phase ½ trial, subjects with localized renal cell carcinoma (tumor 2-4 cm in diameter) will receive ultrasound-guided intratumoral injection of nDoce. As disclosed herein, nDoce is an aqueous suspension of docetaxel particles of at least 95% docetaxel, with a mean particle size (number) of 0.1 microns to 5 microns, an SSA of at least 18 m²/g, and a bulk density (not tapped) of 0.05 g/cm³ to 0.15 g/cm³ to be used in this example. The study will include a dose escalation and dose confirmation phase. During the dose escalation phase, subjects will be enrolled in sequential cohorts within a range of 0.1 to 5 mg/mL of nDoce (with an injection volume equal to 20% of the tumor volume). Subjects will be enrolled in sequential cohorts of three subjects starting at the lowest concentration. Following Data Safety Monitoring Board (DSMB) review of the cohort data, with the exception of the PK data, the DSMB will determine whether to: (a) escalate to the next dose level cohort (no DLT); (b) add three additional subjects to the current cohort (one DLT); (c) if still at the first cohort, stop the study (2 or more DLT); (d) if at higher cohorts, return to the previous (lower) dose cohort and expand by three subjects (more than one DLT). The dose determined to be most suitable for further evaluation, defined as the highest dose with an acceptable safety and tolerability profile (as determined by the DSMB) will enroll additional subjects to total up to 12 subjects at that dose level. Subjects will be evaluated for tumor reduction with imaging of the kidney within a period of time after injection. If a subject shows evidence of tumor growth, the subject will exit the study and receive institutional standard of care (SOC). Any pathology, cytology, or imaging performed within 6 to 9 months of nDoce injection will be collected. Subjects will be evaluated with ultrasound for changes in tumor size once per month for the first 3 months then every other month for a duration of 6 to 9 months. Biopsy may be performed at any time, at the discretion of the Investigator. Plasma samples will be collected on Day 1, before nDoce injection and 1, 2, 4, 6, and 24 hours after injection, as well as at each study visit, to characterize the pharmacokinetics (PK) of nDoce administered as an intratumoral injection.

Objectives: The primary objective is to evaluate the safety and tolerability of nDoce intratumoral injection of renal cell carcinoma. The secondary objectives are to describe the PK of intratumoral injection of nDoce, and to determine whether any of the nDoce concentrations show preliminary signs of efficacy.

Description of the Study Design: Subjects will be enrolled in sequential, escalating cohorts of nDoce intratumoral injection at concentrations selected within a range of 0.1 to 5 mg/mL. The study will include a dose escalation phase and a dose confirmation phase.

Tumor Volume Calculation: Injection volume will be calculated as equal to 20% of the volume of the tumor. The diameter of the tumor lesion will be reported by the radiologist, as measured from the ultrasound reading.

Endpoints: The primary endpoint will be safety and tolerability, as demonstrated by AE, changes in vital signs, laboratory results, and physical examination findings. The secondary endpoints will be concentration of docetaxel in the systemic circulation post-injection (as determined by PK analysis), and tumor response (Eisenhauer et al. New response evaluation criteria in solid tumours: Revised RECIST guideline (version 1.1), European Journal of Cancer 45 (2009 228-247).

Study Drug: The study drug will be supplied in clinical supplies kit. Each kit will contain one vial of docetaxel particles powder and one vial of Sterile Reconstitution Solution. The docetaxel particles powder vial will contain sterile nanoparticulate docetaxel particles at 100 mg/vial appearing as a white powder. The sterile reconstitution solution will contain 1% Polysorbate 80, NF and 8% Ethanol, USP in normal saline solution (0.9% Sodium Chloride for Injection, USP). When ready for use, the docetaxel particles powder will be suspended in the Sterile Reconstitution Solution.

Preparation of Study Drug: An appropriate amount of the reconstitution solution will be added to the docetaxel particles powder vial to reconstitute the drug in suspension to the selected cohort-assigned dose for injection. Once the drug has been reconstituted, the maximum injection volume equal to approximately 20% of the tumor volume per subject will be withdrawn from the vial into a syringe.

Dosing and Administration: Subjects will receive the first cohort-assigned nDoce injection into the base of the tumor within the kidney on Day 1/Visit 2. Intratumoral localization of the injection site will be confirmed by ultrasound prior to tumor injection. If multiple tumors are present, only the largest tumor (not to exceed 4 cm in diameter) will be injected. A 22-gauge needle will be used for injection into the tumor. A total volume, equal to approximately 20% of the tumor volume of nDoce will be injected in increments of approximately 1 cm apart, with up to 8 injections into the tumor. Injections will be performed in a tangential approach grid-like pattern to cover the entire area of the tumor. The investigator will inject up to 5 mm outside of the tumor margin. The total dose administered will not exceed the assigned cohort dose dependent on tumor volume.

Dosing and Dose Escalation Schedule: nDoce will be administered in concentrations based on cohort assignment. Total volume administered will not exceed 20% of the tumor volume in any subject. The study will consist of a dose escalation phase and a dose confirmation phase. During direct injection dose escalation, cohorts will be enrolled sequentially starting at the selected lowest dose. Each cohort will have a planned minimum of three subjects. Escalation to the next cohort will proceed following review of data by the DSMB. All clinical data from subjects in each cohort, including all DLTs described in this section and excluding PK, will be reviewed and evaluated by the DSMB once all three subjects have completed a visit to determine if the dose received is considered safe and tolerable, and to determine if dose escalation may occur. At the initial review, if the DSMB determines cohort 1 is safe (no DLT), escalation to the next dose level, cohort 2, will occur. If ≥1 DLT occurs at cohort 1, three additional subjects will be added to cohort 1. If ≥1 DLT occurs in the additional three subjects, the study will stop. If no additional DLT occurs in the additional three subjects, the study will escalate to the next dose level, cohort 2. Three subjects will be dosed at cohort 2. If the DSMB determines cohort 2 is safe (no DLT), escalation to the next dose level, cohort 3, will occur. If ≥1 DLT occurs at cohort 2, three additional subjects will be added to cohort 2. If ≥1 DLT occurs in the three additional cohort 2 subjects, the study will return to the previous (lower) dose, cohort 1, and proceed to dose confirmation. If no additional DLT occurs in the additional three subjects, the study will escalate to the next dose level, cohort 3. Three subjects will be dosed at cohort 3. If the DSMB determines cohort 3 is safe (no DLT), dose confirmation at cohort 3 will occur. If ≥1 DLT occurs at cohort 3, three additional subjects will be added to cohort 3. If ≥1 DLT occurs in the three additional cohort 3 subjects, the study will return to the previous (lower) dose, cohort 2, and proceed to dose confirmation. If no additional DLT occurs in the additional three subjects, the study will complete enrollment at cohort 3 in dose confirmation. The dose most suitable for further evaluation will be the highest dose with an acceptable safety and tolerability profile as determined by the DSMB. If one or fewer subjects in a six-subject cohort, or no subjects in a three-subject cohort at the highest dose, experience DLT, that cohort will be taken into the dose confirmation phase. If greater than one subject in a six-subject cohort experience DLT, the previous dose will be taken into the dose confirmation phase. Once the dose deemed appropriate for further evaluation has been determined by the DSMB, additional subjects will be enrolled to provide up to a total of 12 subjects dosed at that dose level.

Definition of Dose Limiting Toxicity (DLT): Included in the review of AEs (adverse events) and general study data pertaining to safety (laboratory results, vital signs, physical examination findings) there will be rules for non-escalation. Any AE that is considered related or probably related to nDoce is potentially a DLT. DLTs will, in addition, include the following: 1) Procedure-related events that require hospitalization or surgical intervention and some procedure-related events that require medical intervention; 2) All Grade 3-4 AE which are possibly related to study drug will be considered DLT except: a) Grade 3 nausea or Grade 3-4 vomiting and diarrhea that persist for less than 48 hours in patients who have not received optimal anti-emetic or anti-diarrhea prophylaxis; b) Grade 3 fatigue less than 5 days; c) Grade 3 laboratory abnormalities that are not clinically significant and return to normal (with or without intervention) within 48 hours; 3) Grade 3 thrombocytopenia with clinically significant hemorrhage; 4) Grade 2 toxicity that prevents further treatment or persists for at least 3 weeks; and 5) Any life-threatening event (unless there is a clear alternative explanation that the event is not related to the procedure or the investigational product itself).

Dose Adjustments: As this study is evaluating one administration of nDoce in each subject, with a maximum volume equal to 20% of the tumor volume injected, there will be no dose adjustment or modification in an individual subject for the first injection.

Duration of Therapy: A single administration of nDoce will be injected directly into the tumor on Day 1/Visit 2.

The results of this study will demonstrate the effectiveness of a method of treatment for treating a kidney tumor in a subject comprising directly injecting via intratumoral injection a composition comprising taxane particles into the kidney tumor.

1. A method of treating a kidney tumor in a subject, the method comprising administering an effective amount to treat the kidney tumor of a composition comprising docetaxel particles suspended in a liquid carrier to a kidney tumor of the subject via intratumoral injection, wherein the docetaxel particles comprise at least 98% docetaxel, wherein the docetaxel particles have a mean particle size (number) of from 0.1 microns to 5 microns, and wherein the docetaxel particles have a specific surface area (SSA) of at least 35 m²/g.

2. The method of claim 1, wherein the administering comprises two or more separate administrations.

3. The method of claim 1, wherein the administering comprises two or more separate administrations twice a week for at least one week, and wherein the two or more separate administrations are separated by at least one day.

4-5. (canceled)

6. The method of claim 1, wherein the taxane particles have a mean particle size (number) of from 0.1 microns to 1.5 microns.

7. (canceled)

8. The method of claim 1, wherein the docetaxel particles comprise at least 99% docetaxel.

9-12. (canceled)

13. The method of claim 1, wherein, the docetaxel particles are not bound to, encapsulated in, or coated with one or more of a monomer, a polymer (or biocompatible polymer), a protein, a surfactant, or albumin.

14-15. (canceled)

16. The method of claim 1, wherein the liquid carrier is an aqueous carrier.

17. The method of claim 16, wherein the aqueous carrier comprises a surfactant, and wherein the surfactant is a polysorbate.

18. The method of claim 17, wherein the polysorbate is polysorbate 80, and wherein the polysorbate 80 is present in the liquid carrier at a concentration of about 0.01% w/v to about 1% w/v.

19. The method of claim 1, wherein the composition further comprises a diluent, wherein the liquid carrier and the diluent form a mixture, and wherein the composition is a suspension of the docetaxel particles dispersed in the liquid carrier/diluent mixture.

20. (canceled)

21. The method of claim 1, wherein the concentration of the docetaxel particles in the composition is between about 1 mg/mL and about 40 mg/mL.

22. The method of claim 1, wherein the kidney tumor is benign.

23. The method of claim 1, wherein the kidney tumor is malignant.

24. The method of claim 23, wherein the kidney tumor comprises renal cell carcinoma.

25. The method of claim 1, wherein the administering stimulates the endogenous immune system of the subject resulting in the production of tumoricidal cells and infiltration of the tumoricidal cells in and/or around the tumor site at a level sufficient to treat the tumor.

26. The method of claim 25, wherein the stimulation of the endogenous immune system produces a cellular immune response.

27. The method of claim 25, wherein the stimulation of the endogenous immune system produces a humoral immune response.

28. (canceled)

29. The method of claim 25, wherein the tumoricidal cells comprise dendritic cells, macrophages, T-cells, B cells, lymphocytes, or natural killer (NK) cells, or combinations thereof.

30. (canceled)
